(19) United States
(12) Patent Application Publication
(10) Pub. No.: US 2005/0240051 A1
(43) Pub. Date: Oct. 27, 2005

(54) Dispersing agent or solubilizing agent containing calixarene compound

(75) Inventors: Naoki Yasuda, Kawasaki-shi (JP); Miho Furukawa, Kawasaki-shi (JP)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: Ajinomoto Co., Inc., Tokyo (JP)

(21) Appl. No.: 11/119,290

(22) Filed: Apr. 29, 2005

Related U.S. Application Data
(63) Continuation of application No. PCT/JP03/13751, filed on Oct. 28, 2003.

(30) Foreign Application Priority Data
Oct. 30, 2002 (JP) 316869/2002

(51) Int. Cl. B01J 20/22; C07C 43/20

(52) U.S. Cl. 560/140; 502/401; 568/633

(57) ABSTRACT

The invention provides a dispersant or a solubilizer containing a particular calixarene compound, wherein, of the phenolic hydroxyl groups constituting calixarene, at least one is not substituted, and at least one is substituted by a group having a total carbon number of not less than 10 and comprising one or more alkyleneoxy groups and/or a hydrocarbon group. A carbon-based material (fullerene, carbon nano tube and the like), an organic pigment (phthalocyanine blue and the like), and the like can be dispersed or solubilized in an organic matrix (organic solvent and the like) with the dispersant or solubilizer.

### TECHNICAL FIELD

The present invention relates to a dispersant or a solubilizer for dispersing or solubilizing a carbon-based material such as fullerene, carbon nano tube and the like or an organic pigment such as phthalocyanine pigment, azo pigment, quinacridone pigment, anthraquinone pigment, diketopyrrolopyrrole pigment and the like in an organic matrix such as an organic solvent, resin, lubricating oil, oil paint ink and the like, which is a technique widely usable in the fields of composite materials, lubricants, electrically conductive materials, optical materials and the like.

### BACKGROUND ART

Calixarene, which is a cyclic inclusion compound, can include various compounds and is used as a dispersant or a solubilizer of a guest compound permitting inclusion. Examples of application in an organic matrix include an embodiment wherein a calixarene derivative wherein all phenolic hydroxyl groups are acylated is used as a metal capturing agent in a resin composition (Patent reference 1: JP-T-01-501006), an embodiment wherein, utilizing the markedly high crystallinity of a calixarene derivative wherein a phenolic hydroxyl group is or is not modified, an additive is uniformly dispersed by forming a complex with the additive in the solid (crystal) form (Patent reference 2: JP-A-2001-055469), and the like. In the latter case, the side chain that modifies a hydroxyl group desirably has not more than 10 carbon atoms, which is considered to aim at maintaining the crystallinity of the additive.

As a guest compound that can be included by calixarene, for example, fullerene can be mentioned. Fullerene is a general name of spherical carbon molecules predicted by Oosawa et al. in 1970 (Non-patent reference 1: The Chemical Society of Japan, Quarterly Review of Chemistry, “Chemistry of the third carbon isotope, fullerene”, Japan Scientific Societies Press, Nov. 20, 1999, No. 43, pp. 6-14), and successfully synthesized by Kroto et al. Of them, C₆₀ having a diameter of about 0.7 nm consisting of 60 carbon atoms linked in a soccer ball structure is representative. In view of the specific shape of fullerene, various applications for fullerene such as as a lubricant, bearing and the like, each having a nano size, have been expected from the beginning when it was found, and consideration of applications based on the utilization of the photosensitizing action of fullerene and its property as a semiconductor is underway, as subsequent physicochemical studies of fullerene have proceeded. Production methods include laser irradiation when fullerene was first found, then generation by arc discharge, and a synthetic method comprising, in combination, generation by burning and purification by calixarene that specifically produces a clathrate with fullerene, which has been recently established. At present, industrial adaptation of fullerene is seldom found, and only research and development is underway. However, due to the cost reduction by the above-mentioned mass production and increasing interest in nanotechnology, utilization of fullerene at an industrial level is expected to be realized in the near future.

When the specific performance of fullerene is to be exhibited, and when, for example, fullerene is used as a lubricant and the like utilizing its molecular shape, fullerene molecules are preferably dispersed individually. However, fullerene has a low solubility in various solvents and the like. For example, its solubility in 1-methylnaphthalene (in which it has comparatively high solubility) is about 33 mg/ml at around normal temperature. Its solubility is about 3 mg/ml in toluene, which is a frequently used solvent, and, when it comes to hydrocarbon solvents and alcohol solvents, fullerene is very difficult to dissolve therein with a solubility of not more than 0.1 mg/ml (Non-patent reference 2: Hisanori Shinohara, Yahachi Saito, “Chemistry and physics of fullerene”, first impression of first printing, The University of Nagoya Press, Jan. 15, 1997, p. 40-41).

It is also difficult to monodisperse carbon-based materials, such as carbon nano tube, graphite and the like, other than fullerene, in an organic solvent, resin and the like, and a superior dispersant is desired.

The problem to be solved by the present invention is to provide a dispersant or a solubilizer containing a particular calixarene compound that can better disperse or solubilize, for example, carbon-based materials such as fullerene, carbon nano tube, and the like and organic pigments such as phthalocyanine pigment, azo pigment, quinacridone pigment, anthraquinone pigment, diketopyrrolopyrrole pigment and the like in organic matrices such as organic solvent, resin, lubricating oil and the like, than conventional ones, and furthermore, to provide a lubricant containing these dispersants or solubilizers.

### DISCLOSURE OF THE INVENTION

The present inventors have conducted intensive studies in an attempt to solve the above-mentioned problems. As the reasons for the failure to achieve the satisfactory dispersibility in the above-mentioned Patent reference 2, the present inventors considered two points that (1) since all the phenolic hydroxyl groups of calixarene are substituted by a side chain having not more than 10 carbon atoms and a phenolic hydroxyl group does not exist, the power to adsorb to the substrate surface, which is the dispersion object, is weak, and (2) when the substituent of the phenolic hydroxyl group is a side chain having not more than 10 carbon atoms, the affinity for an organic matrix is not sufficient. Thus, the inventors tried to disperse and solubilize carbon-based materials using a particular calixarene compound free of such causes, namely, a particular calixarene compound, wherein, of the phenolic hydroxyl groups constituting calixarene,
(A) at least one is not substituted, and
(B) at least one is substituted by a group having a total carbon number of not less than 10, which comprises a group comprising one or more alkyleneoxy groups and/or a hydrocarbon group, and surprisingly found that, for example, carbon-based materials such as fullerene, carbon nano tube and the like and organic pigments such as phthalocyanine pigment, azo pigment, quinacridone pigment, anthraquinone pigment, diketopyrrolopyrrole pigment and the like can be better dispersed or solubilized in organic matrices such as organic solvent, resin, lubricating oil and the like than conventional ones, which resulted in the completion of the present invention. In addition, compositions comprising these dispersants or solubilizers are also useful as lubricants.

Accordingly, the present invention provides the following items.

[1] A dispersant or a solubilizer comprising a calixarene compound, wherein, of the phenolic hydroxyl groups constituting calixarene,
(A) at least one is not substituted, and
(B) at least one is substituted by a group having a total carbon number of not less than 10, which comprises a group comprising one or more alkyleneoxy groups and/or a hydrocarbon group (hereinafter to be also referred to as calixarene compound (I)).

[2] The dispersant or solubilizer of the above-mentioned [1], wherein the calixarene compound is represented by the following formula (1) or (2): 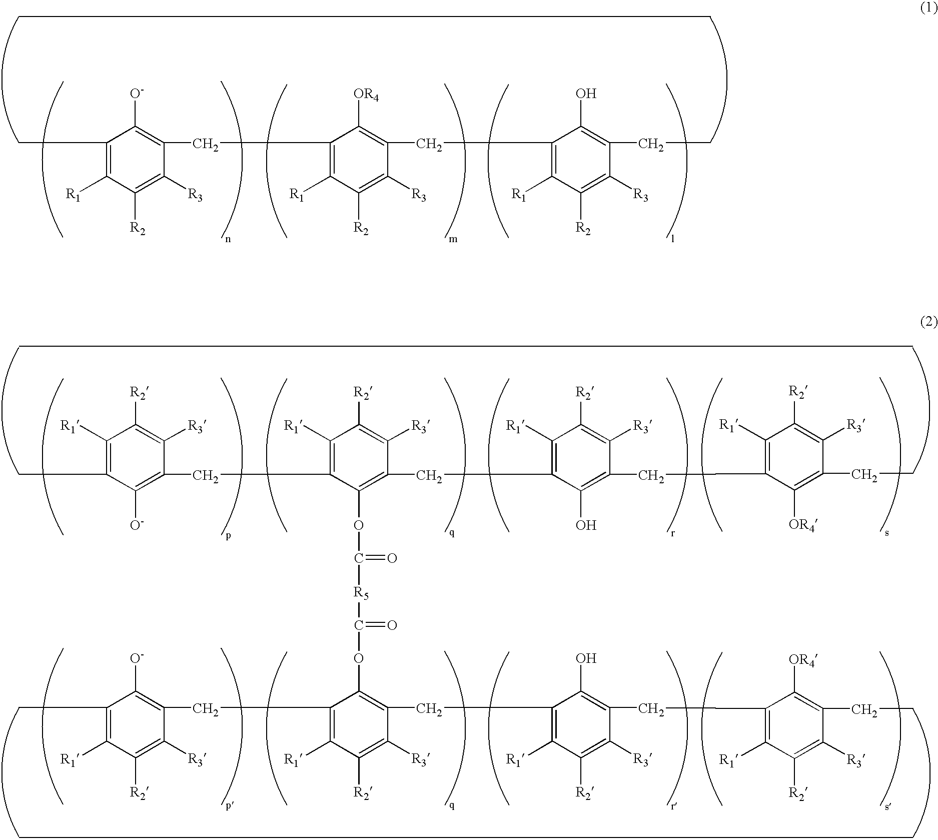
wherein R₁, R₂, R₃, R₁, R₂′ and R₃′ may be the same or different and each is a hydrogen atom, a chain hydrocarbon group optionally having substituent(s), an aryl group optionally having substituent(s), an alkoxy group optionally having substituent(s), a halogen atom, a nitro group, an acyl group, a carboxyl group, a sulfonic acid group or an amino group optionally having substituent(s),
R₁, R₂ and R₃ in the number of n, m or 1 may be the same or different,
R₁′, R₂′ and R₃′ in the number of p, q, r or s may be the same or different,
R₁′, R₂′ and R₃′ in the number of p′, r′ or s′ may be the same or different;
R₄ and R₄′ may be the same or different and each is a C₁₀₋₂₀ alkyl group optionally having substituent(s) or a C₉₋₂₀ alkyl-carbonyl group optionally having substituent(s) or a group represented by the formula (3): —(R₆CO₂)x-R₇, the formula (4): —(R₈O)y-R₉ or the formula (5): —(CO—R₁₀O)w-COR₁₁ (in the formula (3), the formula (4) and the formula (5), R₆, R₈ and R₁₀ may be the same or different and each is a C₁₋₂₀ alkylene group optionally having substituent(s), R₇, R₉ and R₁₁ may be the same or different and each is a hydrogen atom, an acyl group or a C₁₋₂₀ alkyl group optionally having substituent(s), and x, y and w may be the same or different and each is an integer of 1 to 200) (provided that the total carbon number of each of the groups represented by the formula (3), the formula (4) and the formula (5) is not less than 10),
R₄ in the number of m may be the same or different,
R₄′ in the number of s may be the same or different,
R₄′ in the number of s′ may be the same or different;
R₅ is a C₂₋₂₀ alkylene group optionally having substituent(s),
R₅ in the number of q may be the same or different;
n is an integer of 0 to 8, m is an integer of 1 to 9, 1 is an integer of 1 to 9, provided that n+m+1 is an integer of 4 to 10;
p and p′ may be the same or different and each is an integer of 0 to 7, q, r, r′, s and s′ may be the same or different and each is an integer of 1 to 8, provided that p+q+r+s and p′+q+r′+s′ may be the same or different and each is an integer of 4 to 10 (hereinafter the calixarene compounds represented by the formulas (1) and (2) are to be referred to as calixarene compound (1) and calixarene compound (2), respectively).

[3] The dispersant or solubilizer of the above-mentioned [2], wherein R₄ and R₄′ may be the same or different and each is a C₁₀₋₂₀ alkyl group optionally having substituent(s), a C₉₋₂₀ alkyl-carbonyl group optionally having substituent(s) or a group represented by the formula (3): —(R₆CO₂)x-R₇ or the formula (4): —(R₈O)y-R₉ (in the formula (3) and the formula (4), R₆ and R₈ may be the same or different and each is a C₁₋₂₀ alkylene group optionally having substituent(s), R₇ and R₉ may be the same or different and each is a hydrogen atom, an acyl group or a C₁₋₂₀ alkyl group optionally having substituent(s), and x and y may be the same or different and each is an integer of 1 to 200) (provided that the total carbon number of each of the groups represented by the formula (3) and the formula (4) is not less than 10).

[4] The dispersant or solubilizer of the above-mentioned [2] or [3], wherein R₁, R₂, R₃, R₁′, R₂′ and R₃′ may be the same or different and each is a hydrogen atom or a chain hydrocarbon group optionally having substituent(s).

[5] A carbon composite comprising a carbon-based material and a dispersant or solubilizer of any of the above-mentioned [1]-[4].

[6] The carbon composite of the above-mentioned [5], wherein the carbon-based material is any of carbon black, carbon nano tube, graphite, carbon fiber, amorphous carbon and diamond powder, and a dispersant of any of the above-mentioned [1]-[4] acts on the surface of the carbon-based material.

[7] The carbon composite of the above-mentioned [5], wherein the carbon-based material is fullerene and included in a solubilizer of any of the above-mentioned [1]-[4].

[8] An organic pigment composite comprising an organic pigment and a dispersant or solubilizer of any of the above-mentioned [1]-[4].

[9] The organic pigment composite of the above-mentioned [8], wherein the organic pigment is any of a phthalocyanine pigment, an azo pigment, a quinacridone pigment, a diketopyrrolopyrrole pigment and an anthraquinone pigment and a dispersant of any of the above-mentioned [1]-[4] acts on the surface of the organic pigment.

[10] The lubricant comprising a dispersant or solubilizer of any of the above-mentioned [1]-[4].

[11] A calixarene compound represented by the following formula (1′) or (2′): 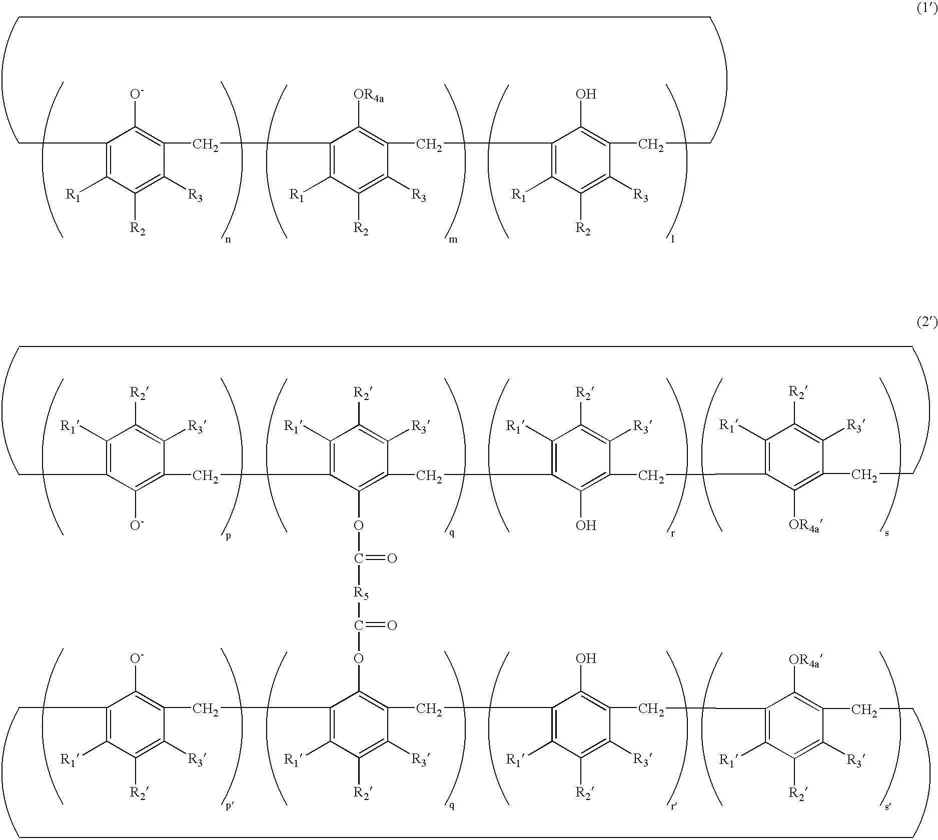
wherein R₁, R₂, R₃, R₁, R₂′ and R₃″ may be the same or different and each is a hydrogen atom, a chain hydrocarbon group optionally having substituent(s), an aryl group optionally having substituent(s), an alkoxy group optionally having substituent(s), a halogen atom, a nitro group, an acyl group, a carboxyl group, a sulfonic acid group or an amino group optionally having substituent(s),
R₁, R₂ and R₃ in the number of n, m or 1 may be the same or different,
R₁′, R₂′ and R₃′ in the number of p, q, r or s may be the same or different,
R₁′, R₂′ and R₃′ in the number of p′, r′ or s′ may be the same or different;
R₄ₐ and R₄ₐ′ may be the same or different and each is a group represented by the formula (3): —(R₆CO₂)x-R₇, the formula (4): —(R₈O)y-R₉ or the formula (5): —(CO—R₁₀O)w-COR₁₁ (in the formula (3), the formula (4) and the formula (5), R₆, R₈ and R₁₀ may be the same or different and each is a C₁₋₂₀ alkylene group optionally having substituent(s), R₇, R₉ and R₁₁ may be the same or different and each is a hydrogen atom, an acyl group or a C₁₋₂₀ alkyl group optionally having substituent(s), and x, y and w may be the same or different and each is an integer of 1 to 200) (provided that the total carbon number of each of the groups represented by the formula (3), the formula (4) and the formula (5) is not less than 10),
R₄ₐ in the number of m may be the same or different,
R₄ₐ′ in the number of s may be same or different,
R₄ₐ′ in the number of s′ may be the same or different;
R₅ is a C₂₋₂₀ alkylene group optionally having substituent(s),
R₅ in the number of q may be the same or different;
n is an integer of 0 to 8, m is an integer of 1 to 9, 1 is an integer of 1 to 9, provided that n+m+1 is an integer of 4 to 10;
p and p′ may be the same or different and each is an integer of 0 to 7, q, r, r′, s and s′ may be the same or different and each is an integer of 1 to 8, provided that p+q+r+s and p′+q+r′+s′ may be the same or different and each is an integer of 4 to 10 (hereinafter the calixarene compounds represented by the formula (1′) and (2′) are also to be referred to as calixarene compound (1′) and calixarene compound (2′), respectively).

[12] The calixarene compound of the above-mentioned [11], wherein R₄ₐ and R₄ₐ′ may be the same or different and each is a group represented by the formula (3): —(R₆CO₂)x-R₇ or the formula (4): —(R₈O)y-R₉ (in the formula (3) and the formula (4), R₆ and R₈ may be the same or different and each is a C₁₋₂₀ alkylene group optionally having substituent(s), R₇ and R₉ may be the same or different and each is a hydrogen atom, an acyl group or a C₁₋₂₀ alkyl group optionally having substituent(s), and x and y may be the same or different and each is an integer of 1 to 200) (provided that the total carbon number of each of the groups represented by the formula (3) and the formula (4) is not less than 10).

### MODE OF EMBODIMENT OF THE INVENTION

The terms used in the present specification are explained below.

The “caprolactone polymer” and “caprolactone ring opened polymer” both refer to a poly-caprolactone (poly-caprolactone) obtained by ring opening polymerization of a caprolactone monomer. The “terminally stearylated caprolactone polymer” is a poly-caprolactone wherein one of the terminals is stearylated, and the “hydroxyl group-terminally stearoylated caprolactone polymer” is a poly-caprolactone wherein the hydroxyl group-terminal is stearoylated.

The “caprolactone polymer chain” is a group wherein one terminal group of poly-caprolactone or a part thereof is liberated, and the “terminally stearylated caprolactone polymer chain” is a poly-caprolactone wherein one of the terminals is stearylated and the other terminal group or a part thereof is liberated. In addition, by “hydroxyl group-terminally stearoylated caprolactone polymer chain” is meant a poly-caprolactone wherein the hydroxyl group-terminal is stearoylated, and the other terminal group or a part thereof is liberated.

The “butyrolactone polymer” and “butyrolactone ring opened polymer” both refer to a poly-butyrolactone (poly-butyrolactone) obtained by ring opening polymerization of a butyrolactone monomer. The “butyrolactone polymer chain” is a group wherein one terminal group of poly-butyrolactone or a part thereof is liberated, and the “terminally stearylated butyrolactone polymer chain” means a polymer wherein one of the terminals is stearoylated, and the other terminal group or a part thereof is liberated.

The definitions of the substituents are explained in detail below.

R₁, R₂, R₃, R₁′, R₂′ and R₃′ may be the same or different and each is a hydrogen atom, a chain hydrocarbon group optionally having substituent(s), an aryl group optionally having substituent(s), an alkoxy group optionally having substituent(s), a halogen atom, a nitro group, an acyl group, a carboxyl group, a sulfonic acid group or an amino group optionally having substituent(s), of which a hydrogen atom and a chain hydrocarbon group optionally having substituent(s) are preferable.

The chain hydrocarbon group optionally having substituent(s) for R₁, R₂, R₃, R₁, R₂′ or R₃′ is, for example, a saturated or unsaturated linear or branched chain hydrocarbon group which preferably has 1-20, more preferably 1-10, carbon atoms and optionally is substituted by the following substituent(s). Examples of the chain hydrocarbon group include alkyl groups such as methyl, ethyl, propyl, isopropyl, butyl, isobutyl, sec-butyl, tert-butyl, pentyl, hexyl, 2-ethylhexyl, octyl, tert-octyl and the like (preferably isopropyl, tert-butyl), alkenyl groups such as allyl, 1-propenyl, 1-butenyl, 1-octenyl and the like, and alkynyl groups such as 1-propynyl, 1-butynyl, 1-octynyl and the like.

As the substituent for the chain hydrocarbon group, for example, a carboxy group, an alkoxycarbonyl group (preferable total carbon number is 2-20, for example, methoxycarbonyl, ethoxycarbonyl, butoxycarbonyl, octyloxycarbonyl and the like), a hydroxy group, a sulfonic acid group, an amino group, an aryl group optionally having substituent(s) (preferable carbon number of aryl moiety is 6-20, preferable substituents are straight chain or branched chain alkyl having 1-12 carbon atoms and the like; for example, phenyl, tolyl, xylyl, p-nonylphenyl and the like) and the like can be mentioned. The chain hydrocarbon group is optionally substituted by one or more of the substituents mentioned above at substitutable positions.

As the chain hydrocarbon group having substituent(s), for example, carboxy-substituted alkyl groups such as carboxymethyl, carboxyethyl, carboxypropyl, carboxybutyl and the like, alkoxycarbonyl-substituted alkyl groups such as methoxycarbonylmethyl, ethoxycarbonylmethyl and the like, hydroxy-substituted alkyl groups such as hydroxymethyl, hydroxyethyl, hydroxypropyl, hydroxybutyl and the like, sulfonic acid-substituted alkyl groups such as sulfomethyl, sulfoethyl, sulfopropyl, sulfobutyl and the like, amino-substituted alkyl groups such as aminomethyl, aminoethyl, aminopropyl, aminobutyl and the like, aryl-substituted alkyl groups such as phenethyl and the like, and the like can be mentioned. Preferable chain hydrocarbon groups optionally having substituent(s) for R₂ or R₂′ are methyl, ethyl, isopropyl, tert-butyl.

The aryl group optionally having substituent(s) for R₁, R₂, R₃, R₁′, R₂′ or R₃′ is, for example, an aryl group preferably having 6-12, more preferably 6-10, carbon atoms, which is optionally substituted by the following substituent(s). Examples of the aryl group include phenyl, naphthyl and the like.

As the substituent for the aryl group, for example, an alkyl group (preferably having 1 to 10 carbon atoms, such as methyl, isopropyl, hexyl, octyl and the like), an aryl group optionally having substituent(s) (preferable total carbon number of aryl moiety is 6-10, preferable substituent is straight chain or branched chain alkyl having 1-12 carbon atoms and the like; such as phenyl, tolyl, xylyl and the like) and the like can be mentioned. The aryl group optionally is substituted by one or more substituents mentioned above at substitutable positions.

As the aryl group optionally having substituent(s), for example, phenyl, tolyl, xylyl, cumenyl, 4-biphenyl and the like can be mentioned, with preference given to phenyl.

Preferable aryl groups optionally having substituent(s) for R₂ or R₂′ are phenyl and tolyl.

The alkoxy group optionally having substituent(s) for R₁, R₂, R₃, R₁′, R₂′ or R₃′ is a linear or branched chain alkoxy group having preferably 1-20, more preferably 1-10, carbon atoms, which is optionally substituted by the following substituent(s). Examples of the alkoxy group include methoxy, ethoxy, propoxy, butoxy, hexyloxy, octyloxy and the like.

As the substituent for the alkoxy group, for example, an alkoxy group (preferable carbon number is 1-4, such as methoxy, ethoxy, propoxy, butoxy and the like) and the like can be mentioned. The alkoxy group is optionally substituted by one or more substituents mentioned above at substitutable positions.

As the alkoxy group optionally having substituent(s), for example, methoxy, ethoxy, propoxy, butoxy, methoxyethoxy, methoxybutoxy and the like can be mentioned.

Preferable alkoxy groups optionally having substituent(s) for R₂ or R₂′ are methoxy and ethoxy.

As the halogen atom for R₁, R₂, R₃, R₁′, R₂′ or R₃′, fluorine atom, chlorine atom, bromine atom and the like can be mentioned.

The acyl group for R₁, R₂, R₃, R₁′, R₂′ or R₃′ is an acyl group having the total carbon number of preferably 2-20, more preferably 2-10. Examples of the acyl group include alkylcarbonyl groups such as acetyl, propionyl, butyryl, hexanoyl, octanoyl, decanoyl and the like, arylcarbonyl groups such as benzoyl and the like, and the like. A preferable acyl group for R₂ or R₂′ is acetyl.

The amino group optionally having substituent(s) for R₁, R₂, R₃, R₁′, R₂′ or R₃′ is an amino group optionally mono- or di-substituted by the following substituent(s). As the substituent for the amino group, for example, an alkyl group (preferable carbon number is 1-4, such as methyl, ethyl, butyl and the like) and the like can be mentioned.

Specific examples of the amino group optionally having substituent(s) include amino, methylamino, ethylamino, dimethylamino, diethylamino, butylamino and the like.

A preferable amino group optionally having substituent(s) for R₂ and R₂′ is dimethylamino.

The “group having the total carbon number of not less than 10, which comprises a group comprising one or more alkyleneoxy groups and/or a hydrocarbon group” in the present invention (hereinafter to be referred to as R₄ group) is a group having a total carbon number of not less than 10, which comprises one or both of a hydrocarbon group and a group comprising one or more alkyleneoxy groups.

The hydrocarbon group includes a linear or branched chain or cyclic saturated or unsaturated hydrocarbon group. The hydrocarbon group is a part of the R₄ group, whose position is not particularly limited and may be the terminal of the R₄ group or any position other than the terminal. The carbon number of the hydrocarbon group is any number that makes the total carbon number of the R₄ group not less than 10.

In the group comprising one or more alkyleneoxy groups, the alkyleneoxy group, which is a constituent unit, is a linear or branched chain, whose carbon number and the condensation degree only need to be a number that makes the total carbon number of the R₄ group not less than 10. The group comprising one or more alkyleneoxy groups also includes an alkyleneoxy group.

As the R₄ group, for example, a C₁₀₋₂₀ alkyl group optionally having substituent(s), a C₉₋₂₀ alkyl-carbonyl group optionally having substituent(s), a group represented by the formula (3): —(R₆CO₂)x-R₇ (wherein each symbol is as defined above, provided that the total carbon number is not less than 10), a group represented by the formula (4): —(R₈O)y-R₉ (wherein each symbol is as defined above, provided that the total carbon number is not less than 10) and the like can be mentioned, and further, a group represented by the formula (5): —(CO—R₁₀O)w-COR₁₁ (wherein each symbol is as defined above, provided that the total carbon number is not less than 10) and the like can be mentioned.

The C₁₀₋₂₀ alkyl group optionally having substituent(s) for R₄ or R₄′, which is one of the R₄ groups, is a linear or branched chain alkyl group optionally substituted by the following substituent(s), and preferably has a carbon number of 12-18. The carbon number of not less than 10 is preferable from the aspect of compatibility with an organic matrix. As the alkyl group, for example, decyl, undecyl, dodecyl, tetradecyl, hexadecyl, stearyl and the like can be mentioned. As the substituent, for example, hydroxy, carboxy, acryloxy, methacryloxy, amino and the like can be mentioned. The alkyl group having a carbon number of 10-20 is optionally substituted by one or more substituents mentioned above at substitutable positions.

Examples of the C₁₀₋₂₀ alkyl group optionally having substituent(s) include decyl, 11-hydroxyundecyl, dodecyl, tetradecyl, hexadecyl, stearyl, 12-hydroxystearyl and the like.

The C₉₋₂₀ alkyl-carbonyl group optionally having substituent(s) for R₄ or R₄′, which is one of the R₄ groups, is an alkyl-carbonyl group wherein the alkyl moiety is a linear or branched chain alkyl group preferably having a carbon number of 14-20, which is optionally substituted by the following substituent(s). The carbon number of the alkyl moiety is preferably not less than 9 from the aspect of compatibility with an organic matrix. As the alkyl-carbonyl group, for example, decanoyl, dodecanoyl, tetradecanoyl, hexadecanoyl, stearoyl and the like can be mentioned. As the substituent, for example, hydroxy and the like can be mentioned. The alkyl-carbonyl group having a carbon number of 9-20 is optionally substituted by one or more substituents mentioned above at substitutable positions.

Examples of the C₉₋₂₀ alkyl-carbonyl group optionally having substituent(s) include decanoyl, dodecanoyl, tetradecanoyl, hexadecanoyl, stearoyl, 12-hydroxystearoyl, with preference given to decanoyl, hexadecanoyl and stearoyl.

The group represented by the formula (3): —(R₆CO₂)ₓ—R₇ (wherein each symbol is as defined above, provided that the total carbon number is not less than 10) for R₄ or R₄′, which is one of the R₄ groups, has the total carbon number of not less than 10 from the aspect of compatibility with an organic matrix. A compound wherein each of R₄ and R₄′ is a group of the formula (3) is a compound wherein a hydroxyl group-terminal of polyester is bonded to a hydroxyl group of calixarene. The hydroxyl group-terminal may or may not be esterified, and in the formula (3), R₇ is a hydrogen atom, an acyl group or a C₁₋₂₀ alkyl group optionally having substituent(s). The C₁₋₂₀ alkyl group optionally having substituent(s) for R₇ is an alkyl group having a carbon number of 1-20, preferably 10-20, which is optionally substituted by the following substituent(s), and, for example, methyl, ethyl, propyl, butyl, hexyl, octyl, decyl, dodecyl, tetradecyl, hexadecyl, stearyl and the like can be mentioned, with preference given to stearyl. As the substituent, for example, hydroxy and carboxy can be mentioned. The C₁₋₂₀ alkyl group optionally having substituent(s) is optionally substituted by one or more substituents mentioned above at substitutable positions. Preferable examples of the C₁₋₂₀ alkyl group optionally having substituent(s) include 12-hydroxystearyl and 1-hexyl-11-carboxyundecyl.

The acyl group for R₇ is an acyl group having the total carbon number of preferably 2-20, more preferably 8-18. Examples of the acyl group include alkylcarbonyl groups such as lauroyl, stearoyl, octanoyl, decanoyl and the like and arylcarbonyl groups such as benzoyl and the like, with preference given to lauroyl and stearoyl.

In the formula (3), the C₁₋₂₀ alkylene group optionally having substituent(s) for R₆ is a straight chain or branched chain alkylene having 1-20, preferably 2-18, carbon atoms, which is optionally substituted by the following substituents. As the alkylene group, for example, methylmethylene, trimethylene, pentamethylene, undecamethylene, heptadecamethylene and the like can be mentioned. As the substituent, hydroxy, carboxy and the like can be mentioned. The alkylene group having a carbon number of 1-20 is optionally substituted by one or more substituents mentioned above at substitutable positions. Examples of the C₁₋₂₀ alkylene group optionally having substituent(s) include methylmethylene, trimethylene, pentamethylene, undecamethylene, heptadecamethylene and the like, with preference given to pentamethylene.

In the formula (3), x is an integer of 1-200, preferably 1-100, more preferably 3-20, from the aspects of compatibility with an aqueous matrix and easy availability of starting materials.

As a preferable group represented by the formula (3), for example, terminally stearylated caprolactone polymer chain, terminally laurylated caprolactone polymer chain, terminally stearylated butyrolactone polymer chain, terminally stearylated hydroxystearic acid polycondensation chain (both having a degree of polymerization x) and the like can be mentioned.

The group represented by the formula (4): —(R₈O)_{y}—R₉ (wherein each symbol is as defined above, provided that the total carbon number is not less than 10) for R₄ or R₄′, which is one of the R₄ groups, has a total carbon number of not less than 10 from the aspect of compatibility with an organic matrix. In the formula (4), R₉ is a hydrogen atom, an acyl group or a C₁₋₂₀ alkyl group optionally having substituent(s). The C₁₋₂₀ alkyl group optionally having substituent(s) for R₉ is an alkyl group having a carbon number of 1-20, preferably 10-20, which is optionally substituted by the following substituent(s), and, for example, methyl, ethyl, propyl, butyl, hexyl, octyl, decyl, dodecyl, tetradecyl, hexadecyl, stearyl and the like can be mentioned, with preference given to decyl, dodecyl, tetradecyl, hexadecyl and stearyl. As the substituent, for example, hydroxy, carboxy and the like can be mentioned. The alkyl group having a carbon number of 1-20 is optionally substituted by one or more substituents mentioned above at substitutable positions. As a preferable example of the C₁₋₂₀ alkyl group optionally having substituent(s), 12-hydroxystearyl can be mentioned.

The acyl group for R₉ is an acyl group having a total carbon number of preferably 2-20, more preferably 8-18. Examples of the acyl group include alkylcarbonyl groups such as lauroyl, stearoyl, octanoyl, decanoyl and the like, and arylcarbonyl groups such as benzoyl and the like can be mentioned, with preference given to lauroyl and stearoyl.

In the formula (4), the C₁₋₂₀ alkylene group optionally having substituent(s) for R₈ is a linear or branched chain alkylene group having a carbon number of 1-20, preferably 1-4, which is optionally substituted by the following substituent(s). As the alkylene group, for example, methylene, ethylene, propylene, dimethylpropylene, butylene and the like can be mentioned. As the substituent, hydroxy, carboxy and the like can be mentioned. The alkylene group having a carbon number of 1-20 is optionally substituted by one or more substituents mentioned above at substitutable positions. Examples of the C₁₋₂₀ alkylene group optionally having substituent(s) include methylene, ethylene, propylene, dimethylpropylene, butylene, hydroxypropylene, bis(hydroxymethyl)propylene and the like, with preference given to ethylene, propylene and hydroxypropylene.

In the formula (4), y is an integer of 1-200, preferably 5-100, more preferably 10-50, from the aspects of compatibility with an aqueous matrix and easy availability of starting materials.

Preferable groups represented by the formula (4) are, for example, terminally stearoylated polyethylene glycol chain, terminally stearylated polyethylene glycol chain, terminally stearoylated polypropylene glycol chain (for each of which the degree of polymerization is y) and the like.

The group represented by the formula (5): —(CO—R₁₀O)_{w}—COR₁₁ (wherein each symbol is as defined above, provided that the total carbon number is not less than 10) for R₄ or R₄′, which is one of the R₄ groups, has a total carbon number of not less than 10 from the aspect of compatibility with an organic matrix. A compound wherein each of R₄ and R₄′ is a group of the formula (5) is one wherein polyester having one terminal modified with carbonyl group is bonded to the hydroxyl group of calixarene. In the formula (5), R₁₁ is a hydrogen atom, an acyl group or a C₁₋₂₀ alkyl group optionally having substituent(s). The C₁₋₂₀ alkyl group optionally having substituent(s) for R₁₁ is an alkyl group having a carbon number of 1-20, preferably 10-20, which is optionally substituted by the following substituent(s). For example, methyl, ethyl, propyl, butyl, hexyl, octyl, decyl, dodecyl, tetradecyl, hexadecyl, stearyl and the like can be mentioned, with preference given to stearyl. As the substituent, for example, hydroxy and carboxy can be mentioned. The C₁₋₂₀ alkyl group optionally having substituent(s) is optionally substituted by one or more substituents mentioned above at substitutable positions. As a preferable example of the C₁₋₂₀ alkyl group optionally having substituent(s), 12-hydroxystearyl can be mentioned.

The acyl group for R₁₁ is an acyl group having a total carbon number of preferably 2-20, more preferably 8-18. Examples of the acyl group include alkylcarbonyl groups such as lauroyl, stearoyl, octanoyl, decanoyl and the like, and arylcarbonyl groups such as benzoyl and the like, with preference given to lauroyl and stearoyl.

In the formula (5), the C₁₋₂₀ alkylene group optionally having substituent(s) for R₁₀ is a linear or branched chain alkylene group having a carbon number of 1-20, preferably 2-18, which is optionally substituted by the following substituent(s). As the alkylene group, for example, methylmethylene, trimethylene, pentamethylene, undecamethylene, heptadecamethylene and the like can be mentioned. As the substituent, hydroxy, carboxy and the like can be mentioned. The alkylene group having a carbon number of 1-20 is optionally substituted by one or more substituents mentioned above at substitutable positions. Examples of the C₁₋₂₀ alkylene group optionally having substituent(s) include methylmethylene, trimethylene, pentamethylene, undecamethylene, heptadecamethylene and the like, with preference given to pentamethylene.

In the formula (5), w is an integer of 1-200, preferably 1-100, more preferably 3-20, from the aspects of compatibility with an aqueous matrix and easy availability of starting materials.

As a preferable group represented by the formula (5), for example, hydroxyl group-terminally stearoylated caprolactone polymer, hydroxyl group-terminally lauroylated caprolactone polymer, hydroxyl group-terminally stearoylated butyrolactone polymer, hydroxyl group-terminally stearoylated hydroxystearic acid condensation polymer (each having a degree of polymerization of w) and the like can be mentioned.

In the formula (1), R₄ in the number of m may be the same or different.

In the formula (2), R₄′ in the number of s may be the same or different, and R₄′ in the number of s′ may be the same or different.

The C₂₋₂₀ alkylene group optionally having substituent(s) for R₅ is a linear or branched chain alkylene group optionally substituted by the following substituent(s), and a preferable carbon number is 4-10. As the alkylene group, for example, tetramethylene, hexamethyleneoctamethylene, decamethylene, dodecamethylene, tetradecamethylene, hexadecamethylene and the like can be mentioned. As the substituent, hydroxy, carboxy and the like can be mentioned. The C₂₋₂₀ alkylene group is optionally substituted by one or more substituents mentioned above at substitutable positions. Preferable examples of the C₂₋₂₀ alkylene group optionally having substituent(s) include tetramethylene, hexamethylene, octamethylene, decamethylene, dodecamethylene, tetradecamethylene, hexadecamethylene, 11-hydroxyhexadecamethylene and the like.

In the formula (2), R₅ in the number of q may be the same or different.

In the formula (1), n is an integer of 0-8, preferably 0-4, m is an integer of 1-9, preferably 2-6, and 1 is an integer of 1-9, preferably 2-6, provided that n+m+1 is an integer of 4-10, preferably 4-8.

The unit to which R₄ is bonded needs to be always present for compatibility with an organic matrix, and m is equal to or greater than 1. In addition, the unit where a hydroxyl group is present needs to be always present to improve the adsorbability of calixarene compound (I) in a dispersion object; namely, 1 is at least 1. A unit where phenoxy ion is present, which is a unit other than these, is produced in the synthetic process to be mentioned below, and whether or not it is present does not prevent the object of the present invention.

In the formula (1), R₁, R₂ and R₃ in the number of n, m or 1 may be the same or different.

In the formula (2), p and p′ may be the same or different, and each is an integer of 0-7, preferably 0-4, and q, r, r′, s and s′ may be the same or different, and each is an integer of 1-8, preferably 2-6, provided that p+q+r+s and p′+q+r′+s′ may be the same or different and each is an integer of 4-10, preferably 4-8.

The unit wherein calixarenes are bonded by an —O—CO—R₅—CO—O— group needs to be always present to bond calixarenes to each other; namely, q is at least 1.

The unit to which R₄′ is bonded thereto needs to be always present for compatibility with an organic matrix, namely, s and s′ are each at least 1. In addition, a unit wherein a hydroxyl group is present needs to be always present to improve adsorbability of calixarene compound (I) to a dispersion object; namely, r and r′ are at least 1. A unit where a phenoxy ion is present, which is a unit other than these, is produced in the synthetic process to be mentioned below, and whether or not it is present does not prevent the object of the present invention.

In the formula (2), R₁′, R₂′ and R₃′ in the number of p, q, r or s may be the same or different.

R₁′, R₂′ and R₃′ in the number of p′, r′ or s′ may be the same or different.

Calixarene in the present invention is a cyclic oligomer wherein phenols optionally having substituent(s) are bonded via a methylene group at the meta-position. Calixarene wherein a methylene group is substituted is encompassed in the scope of the present invention. For example, a cyclic oligomer represented by the formula 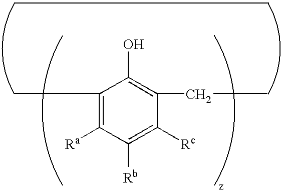
wherein R^{a}, R^{b} and R^{c} may be the same or different and each is a hydrogen atom, a chain hydrocarbon group optionally having substituent(s), an aryl group optionally having substituent(s), an alkoxy group optionally having substituent(s), a halogen atom, a nitro group, an acyl group, a carboxyl group, a sulfonic acid group or an amino group optionally having substituent(s) (each group is as defined for R₁), and z is an integer of 1 to 10 (preferably an integer of 4 to 10)) can be mentioned.

The calixarene compound (I) of the present invention is a compound wherein, of the phenolic hydroxyl groups constituting calixarene,
(A) at least one is not substituted,
(B) at least one is substituted by a group having a total carbon number of not less than 10, which comprises a group comprising one or more alkyleneoxy groups and/or a hydrocarbon group, and a particular calixarene compound (I) having the characteristics of the present invention is effective as a dispersant or a solubilizer.

As the calixarene compound (I) of the present invention, for example, calixarene compound (1) and calixarene compound (2) can be mentioned.

As the calixarene compound (1), calixarene compound (1′) can be mentioned, and calixarene compound (1′) is calixarene compound (1) wherein R₄ is limited to a group selected from the groups represented by the formula (3) to the formula (5). As the calixarene compound (2), calixarene compound (2′) can be mentioned, and calixarene compound (2′) is a calixarene compound (2) wherein R₄ is limited to a group selected from the groups represented by the formula (3) to the formula (5). Therefore, the terms of the formulas (1′) and (2′) are as defined for the corresponding terms in the formulas (1) and (2).

As the calixarene compound (1) and calixarene compound (1′), an embodiment wherein n+m+1 is 6 or 8 is preferable, and an embodiment wherein n is 0, m is 2-4, R₂ is a chain hydrocarbon group optionally having substituent(s) (particularly preferably tert-butyl group) and R₁ and R₃ are hydrogen atoms is more preferable, from the aspect of easy production. In addition, an embodiment wherein n is 0, m is 2-4, and in the case of calixarene compound (1), R₄ is a C₉₋₂₀ alkyl-carbonyl group optionally having substituent(s) or a group represented by the formula (5) (particularly hydroxyl group-terminally stearoylated caprolactone polymer chain), and in the case of calixarene compound (1′), R₄ₐ is a group represented by the formula (5) (particularly hydroxyl group-terminally stearoylated caprolactone polymer chain), is particularly preferable, from the aspect of dispersibility.

As the calixarene compound (2) and calixarene compound (2′), an embodiment wherein p+q+r+s and p′+q+r′+s′ may be the same or different and each is 6 or 8 is preferable, and an embodiment wherein p and p′ are each 0, s and s′ are each 2-4, R₂′ is a chain hydrocarbon group optionally having substituent(s) (particularly preferably tert-butyl group) and each of R₁′ and R₃′ is a hydrogen atom is more preferable, from the aspect of easy production. In addition, an embodiment wherein p and p′ are each 0, s and s′ are each 2-4, and in the case of calixarene compound (2), R₄′ is a C₉₋₂₀ alkyl-carbonyl group optionally having substituent(s) or a group represented by the formula (5) (particularly hydroxyl group-terminally stearoylated caprolactone polymer chain), and in the case of calixarene compound (2′), R₄ₐ′ is a group represented by the formula (5) (particularly hydroxyl group-terminally stearoylated caprolactone polymer chain), is particularly preferable, from the aspect of dispersibility.

The production methods of particular calixarene compound (I) of the present invention are explained below by taking calixarene compound (1) and calixarene compound (2) as examples. When formulas are used for explanation, each symbol in the formulas is as defined above, unless otherwise specified.

Calixarene Compound (1)

First, the phenolic hydroxyl group of calix(u)arene is phenoxy ionized and dissolved in an organic solvent. Phenoxy ionization is generally performed using a base. The symbol u in the calix(u)arene used as a starting material means an integer of 4 to 10, and specific examples of calix(u)arene include 4-tert-butylcalix(8)arene, 4-tert-butylcalix(6)arene, 4-tert-butylcalix(4)arene and the like, with preference given to 4-tert-butylcalix(8)arene. For calix(u)arene, commercially available products may be used or can be also produced by known methods, for example, following or according to the method disclosed in C. D. Gutshe et al., Calixarenes, 4. The synthesis, Characterization, and properties of Calixarenes from p-tert-butylphenol, Journal of American Chemical Society, Vol. 103, No. 13, pp. 3782-3792 (1981).

The organic solvent used for phenoxy ionization is not particularly limited, and, for example, alcohols such as methanol, ethanol, propanol and the like, ketones such as acetone, methyl ethyl ketone and the like, esters such as ethyl acetate, butyl acetate and the like, aromatic hydrocarbons such as toluene, xylene and the like, aliphatic hydrocarbons such as hexane, heptane and the like, tetrahydrofuran (THF), dimethyl sulfoxide (DMSO), dimethylformamide (DMF) and the like can be mentioned, with preference given to methanol, acetone and THF. While the amount of the organic solvent to be used is not particularly limited, it is generally 5-100 parts by weight, preferably 10-50 parts by weight per part by weight of calix(u)arene.

For example, the base to be used for phenoxy ionization is not particularly limited as long as it can phenoxy ionize calix(u)arene and includes, for example, alkali metals such as K, Na and the like, alkali metal hydroxides such as KOH, NaOH and the like, alkali metal carbonates such as Na₂CO₃, K₂CO₃ and the like, alkali metal hydrides such as NaH and the like, organic amines such as aqueous ammonia, triethylamine, diethylamine, ethylenediamine, (C₂H₅)₄N⁺OH⁻ and the like, with preference given to NaH, NaOH and triethylamine. The amount of the base to be used is generally 1-20 molar equivalents, preferably 2-10 molar equivalents, per 1 mol of calix(u)arene.

While the phenoxy ionization varies depending on the solvent to be used, it is preferably performed generally at room temperature−reflux temperature of the solvent to be used, wherein the temperature is raised from room temperature and maintained at reflux temperature for a while and ceased.

After completion of the phenoxy ionization, the product can be isolated or purified by conventional methods such as filtration, concentration, drying and the like, or can be applied to the next step without isolation and purification.

Next, the R₄ group is introduced into the phenoxy ion of a calix(u1+u2)arene compound obtained by phenoxy ionization. With respect to the calix(u1+u2)arene compound, and with reference to the phenolic hydroxyl groups, u1 means not phenoxy ionized, u2 means phenoxy ionized, and u1+u2 is an integer of 4-10.

The reagent to be used for the introduction of the R₄ group is not particularly limited as long as it can react with the phenoxy ion of the calix(u1+u2)arene compound and halides such as R₄Br, R₄Cl, R₄I and the like which are preferable from the aspect of reactivity. The introduction of R₄ group can be performed by, for example, reacting the calix(u1+u2)arene compound with a halide. The introduction of the R₄ group is generally performed in an organic solvent, and, for example, solvents similar to those used for phenoxy ionization can be mentioned, with preference given to acetone and THF. The amount thereof to be used is generally 5-100 parts by weight, preferably 10-50 parts by weight, per part by weight of calix(u1+u2)arene compound (in the case of R₄ introduction without isolation after phenoxy ionization, the amount of use is based on the yield of calix(u1+u2)arene compound obtained quantitatively from calix(u)arene used).

The amount of halide to be used is generally 1-9 mol, preferably 2-6 mol, per 1 mol of calix(u1+u2)arene compound (in the case of R₄ introduction without isolation after phenoxy ionization, the amount of use is based on the yield of calix(u1+u2)arene compound obtained quantitatively from calix(u)arene used).

While the introduction of the R₄ group varies depending on the solvent to be used, it is preferably performed generally at room temperature-reflux temperature of the solvent to be used, wherein the temperature is raised from room temperature and maintained at reflux temperature for a while and ceased.

For preparation of the halide, a suitable method is determined depending on R₄.

For example, a halide wherein R₄ is a C₁₀₋₂₀ alkyl group optionally having substituent(s) can be prepared by reacting an alcohol represented by the formula: R₄₁OH wherein R₄₁ is a C₁₀₋₂₀ alkyl group optionally having substituent(s) with a halogenating reagent (e.g., PCl₃, PBr₃ and the like).

For example, a halide wherein R₄ is a C₉₋₂₀ alkyl-carbonyl group optionally having substituent(s) can be prepared by reacting carboxylic acid represented by the formula: R₄₂CO₂H wherein R₄₂ is a C₉₋₂₀ alkyl group optionally having substituent(s) with a halogenating reagent (e.g., thionyl halide such as thionyl chloride etc., and the like).

For example, a halide wherein R₄ is a group represented by the formula (3): —(R₆CO₂)ₓ—R₇ can be prepared by reacting a compound represented by the formula: HO—(R₆CO₂)ₓ—R₇ with a halogenating reagent (e.g., PCl₃, PBr₃ and the like). As the compound represented by the formula: HO—(R₆CO₂)ₓ—R₇, lactic acid condensate, butyrolactone ring opened polymer, caprolactone ring opened polymer, 12-hydroxydodecane acid condensate, 12-hydroxystearic acid condensate and the like can be mentioned.

For example, a halide wherein R₄ is a group represented by the formula (4): —(R₈O)_{y}—R₉ can be prepared by reacting a compound represented by the formula: R₉—(OR₈)_{y}—OH with a halogenating reagent (e.g., PCl₃, PBr₃ and the like). As the compound represented by the formula: R₉—(OR₈)_{y}—OH, polyethylene glycol monostearyl ether, polypropylene glycol monolauryl ether, polyethylene glycol monostearate, polyethylene glycol monolaurate and the like can be mentioned.

The calixarene compound (I) wherein R₄ is a group represented by the formula (4) can be also produced by directly reacting ethyleneoxide or propyleneoxide with calixarene, beside such a method using a halogenating reagent.

For example, a halide wherein R₄ is a group represented by the formula (5): —(CO—R₁₀O)_{w}—COR₁₁ can be prepared by reacting a group represented by the formula: HO—(CO—R₁₀O)_{w}—COR₁₁ with a halogenating reagent (e.g., thionyl halide such as thionyl chloride etc., and the like). As a compound represented by the formula: HO—(CO—R₁₀O)_{w}—COR₁₁, lactic acid condensate, butyrolactone ring opened polymer, caprolactone ring opened polymer, 12-hydroxydodecane acid condensate, 12-hydroxystearic acid condensate and the like can be mentioned.

After the completion of introduction of the R₄ group, an acid such as acetic acid, dil. hydrochloric acid and the like is added to perform neutralization, whereby unreacted phenoxy ion can be converted to phenol. Without this neutralization treatment, phenoxy ion remains, and cation corresponding to the base used for the phenoxy ionization reaction remains as a counter ion.

In addition, the alkali metal salt of halogen produced by the reaction and the salt resulting from the neutralization treatment can be removed by filtering the reaction mixture.

Calixarene Compound (2)

First, the phenolic hydroxyl group of calixarene is phenoxy ionized and dissolved in an organic solvent. Phenoxy ionization is generally performed using a base.

The organic solvent and base used for the phenoxy ionization may be those similar to the ones used for the production of calixarene compound (1), and the amount of use thereof falls within the similar range. The reaction conditions for phenoxylation may be those similar to the ones used for calixarene compound (1).

After completion of the phenoxy ionization, the product can be isolated and purified by conventional methods such as filtration, concentration, drying and the like, or can be applied to the next step without isolation and purification.

Next, the R₄ group and R₅ group are introduced into the phenoxy ion of a calix(u1+u2)arene compound. Introduction of the R₄ group and R₅ group may be performed independently, but simultaneous introduction is efficient and preferable. In the case of separate introductions, the order of introduction is not particularly limited. Introduction of the R₄ group and R₅ group is generally performed in a solvent, and, for example, the solvents similar to the ones used for phenoxylation step of calixarene compound (1) can be mentioned, with preference given to acetone and THF. The amount of the solvent to be used is generally 5-100 parts by weight, preferably 10-50 parts by weight, per part by weight of calix(u1+u2)arene compound (in the case of R₄ introduction without isolation after phenoxy ionization, the amount of use is based on the yield of calix(u1+u2)arene compound obtained quantitatively from calix(u)arene used).

Introduction of the R₄ group into the calixarene compound can be performed in the same manner as in calixarene compound (1). Introduction of the R₅ group is performed using a reagent capable of reacting with the phenoxy ion of calix(u1+u2)arene compound, and, from the aspect of reactivity, dihalides such as Br—C(═O)—R₅—C(═O)—Br, Cl—C(═O)—R₅—C(═O)—Cl, I—C(═O)—R₅—C(═O)—I and the like are preferable.

The amount of dihalide to be used is generally 1-8 mol, preferably 2-6 mol, per 1 mol of calix(u1+u2)arene compound (in the case of introduction of the R₄ group and R₅ group without isolation after phenoxy ionization, the amount of use is based on the yield of calix(u1+u2)arene compound obtained quantitatively from calix(u)arene used).

While the introduction of the R₄ group and R₅ group varies depending on the solvent to be used, it is preferably performed generally at room temperature−reflux temperature of the solvent to be used, wherein the temperature is raised from room temperature and maintained at reflux temperature for a while and ceased.

The calixarene compound (2) may be produced as a by-product during production of calixarene compound (1).

After the completion of introduction of the R₄ group and R₅ group, neutralization treatment is applied in the same manner as in calixarene compound (1).

In addition, the alkali metal salt of halogen produced by the reaction and the salt resulting from the neutralization treatment can be removed by filtering the reaction mixture.

The calixarene compound (I) of the present invention is particularly highly useful for dispersing or dissolving a carbon-based material and an organic pigment in an organic matrix. The carbon-based material in the present invention is a substance consisting only of a carbon atom, and, for example, carbon black, carbon nano tube, graphite, carbon fiber, amorphous carbon, diamond powder and fullerene can be mentioned. The calixarene compound (I) of the present invention can be preferably applied using a carbon fiber and fullerene. The organic pigment in the present invention is an organic compound having a conjugated electron, which develops color in visible light region, and, for example, phthalocyanine pigments (e.g., phthalocyanine blue, phthalocyanine green, basic phthalocyanine blue and the like), azo pigments such as azo, diazo, condensated azo and the like (e.g., pigment red 3, pigment red 21, pigment red 144, pigment orange 5, pigment orange 38, pigment brown 25, pigment yellow 1, pigment yellow 12 and the like), quinacridone pigments (e.g., pigment violet 19, pigment red 207, pigment red 206 and the like), anthraquinone pigments (e.g., pigment yellow 24, pigment orange 40, pigment red 177, pigment blue 6 and the like), thio indigo pigments (e.g., pigment red 88 and the like), indanthrone pigments (e.g., pigment blue 60, pigment blue 64 and the like), isoindolinone pigments (e.g., pigment yellow 109, pigment orange 61 and the like), diketopyrrolopyrrole pigments (chloride, methylide, dimethylamino compound and the like of diketopyrrolopyrrole) and the like can be mentioned, with preference given to phthalocyanine pigments, azo pigments, quinacridone pigments, diketopyrrolopyrrole pigments and anthraquinone pigments. The calixarene compound (I) of the present invention can be preferably applied using phthalocyanine blue. The organic matrix is a liquid or solid dispersion medium mainly composed of an organic product, in which the above-mentioned carbon-based material is dispersed and, for example, organic solvent, resin, lubricating oil, oil paint ink and the like can be mentioned.

A carbon composite can comprise (a) a dispersant or a solubilizer containing the calixarene compound (I) of the present invention and (b) a carbon-based material. The carbon composite is a complex compound of calixarene compound (I) and a carbon-based material. The carbon-based material can be dispersed or dissolved in an organic matrix by forming a carbon composite with calixarene compound (I). When the carbon-based material is any of carbon black, carbon nano tube, graphite, carbon fiber, amorphous carbon and diamond powder, the dispersant of the present invention acts on the surface of these carbon-based materials and can disperse the carbon-based material in an organic matrix.

When the carbon-based material is fullerene, the solubilizer of the present invention includes fullerene, and fullerene can be dissolved in an organic matrix.

As the production method of the carbon composite of the present invention, dry treatment methods comprising adding calixarene compound (I) to a carbon-based material and mixing by stirring in a high speed mixer such as Henschel mixer and the like, wet treatment methods comprising dissolving calixarene compound (I) in a solvent, adding a carbon-based material, mixing, filtration or solvent evaporation and the like can be mentioned, with preference given to wet treatment methods since the carbon-based material is very small. The solvent to be used for the wet treatment methods is not particularly limited as long as it can dissolve calixarene compound (I), and, for example, the same solvents as used for the production of calixarene compound (1) and calixarene compound (2) can be mentioned. For mixing in the wet treatment methods, a mixer using a mixing medium such as a ball mill, a sandmill and the like, and the like can be used besides a mixing impeller, and heat, ultrasonics and the like may be applied for the purpose of increasing the treatment efficiency.

In addition, integral blend methods wherein calixarene compound (I) is simultaneously added when a carbon-based material or an organic pigment is added to an organic matrix, and a surface-treatment is conducted while dispersing a carbon-based material or an organic pigment may be employed without any problem.

For forming a composite of fullerene and calixarene compound (I), for example, fullerene is preferably dissolved in a solvent such as toluene, dichlorobenzene and the like, calixarene compound (I) dissolved in advance in a solvent is added thereto, and the solvent is evaporated. By forming a composite of fullerene by this method, fullerene can be dissolved in a solvent in which non-composite fullerene cannot be dissolved.

An organic pigment composite comprising a dispersant or a solubilizer, comprising the calixarene compound (I) of the present invention, and an organic pigment is a compound comprising a composite of calixarene compound (I) and an organic pigment. An organic pigment can be dispersed or dissolved in an organic matrix in the form of an organic pigment composite with calixarene compound (I). When the organic pigment is phthalocyanine, azo, quinacridone, anthraquinone or diketopyrrolopyrrole pigment, the dispersant of the present invention acts on the surface of these organic pigments and can disperse an organic pigment in an organic matrix.

The organic pigment composite of the present invention can be produced in the same manner as in the case of a carbon-based material composite. Since efficiency of the surface treatment is superior, wet treatment methods are more suitable. The solvent that can be used for the wet treatment methods is not particularly limited as long as it can dissolve calixarene compound (I), and, for example, the solvents used for the production of calixarene compound (1) and calixarene compound (2) can be mentioned.

A composite of phthalocyanine blue and calixarene compound (I) can be preferably formed by, for example, adding phthalocyanine blue and calixarene compound (I) to a solvent such as toluene, methyl ethyl ketone, hexane and the like, and mixing and dispersing them with a ball mill and the like. By forming a composite of phthalocyanine blue by this method, phthalocyanine blue can be dissolved in a solvent in which non-composite phthalocyanine blue cannot be dissolved.

The dispersant or solubilizer of the present invention is also useful for lubricants.

Moreover, the dispersant or solubilizer of the present invention can be also applied to magnetic materials such as ferrite and the like, electrically conductive materials such as copper powder, nickel powder and the like, flame-retardants such as magnesium hydroxide, ammonium polyphosphate and the like, and the like, besides the carbon-based material and pigment (organic pigment).

The effects achieved by the present invention are considered to be expressed by the following mechanisms.

The calixarene skeleton of the calixarene compound (I) of the present invention is a structure wherein a number of benzene rings are bonded in a cyclic form, and shows high affinity for carbon-based materials having a structure wherein a benzene ring is similarly condensed, due to a π-π interaction. In addition, since a calixarene wherein a phenolic hydroxyl group remains unreacted is more superior in terms of dispersibility and solubilizability, this hydroxyl group is considered to be conducive to the improved affinity. Particularly, since fullerene has a size fitting in the inside of the cyclic structure of calixarene (I), one fullerene molecule is included in one calixarene compound (I) to give a composite at a molecular level. Furthermore, many of the organic pigments such as phthalocyanine blue and the like generally have plural benzene rings, which interact with the benzene rings of calixarene compound (I), thus affording efficient adsorption on the pigment surface.

On the other hand, since the calixarene compound (I) of the present invention has a hydrocarbon chain having affinity for an organic matrix, the carbon composite and organic pigment composite of the present invention have improved affinity for these matrices, and show improved solubility and dispersibility.

### EXAMPLES

The contents of the present invention are now explained in detail by referring to Examples and Comparative Examples, which are intended to not limit the invention but clearly show the contents of the present invention. Note that “parts” means “parts by weight”.

### Example 1

Synthesis of Compound (1)

4-tert-Butylcalix(8)arene (50.00 parts, manufactured by Kawaguchi Chemical Co., Ltd.) and methanol (791.00 parts, manufactured by JUNSEI CHEMICAL CO., LTD.) were collected in a beaker, and, after thorough stirring, a 1 mol/L-sodium hydroxide (manufactured by JUNSEI CHEMICAL CO., LTD.)/methanol solution (96.15 parts) was added, and the mixture was stirred at room temperature for 24 hr. After the completion of the stirring, the mixture was filtered using a Kiriyama funnel and a paper filter (No. 5C), and a filter cake was dried under reduced pressure (120° C., 6 hr).

A reaction product (5.00 parts) of the above-mentioned 4-tert-butylcalix(8)arene and sodium hydroxide and THF (35.48 parts, manufactured by JUNSEI CHEMICAL CO., LTD.) were placed in a flask equipped with a mixer, and the mixture was stirred at room temperature. 1-Bromooctadecane (2.47 parts, 2-fold mol relative to 4-tert-butylcalix(8)arene, manufactured by Tokyo Kasei Kogyo Co., Ltd.) was placed in a dropping funnel, set on the flask with a distillation column, and 1-bromooctadecane was added dropwise over 15 min with stirring. After the completion of the dropwise addition, the dropping funnel was washed with THF (8.87 parts) and refluxed for 3 hr.

After the completion of refluxing, the mixture was allowed to cool and neutralized by adding acetic acid (manufactured by JUNSEI CHEMICAL CO., LTD.) into the flask. Then, the precipitate was removed with a Kiriyama funnel and a paper filter (No. 5C), and the filtrate was passed through a syringe filter (0.2 μm) to remove fine precipitate. The solvent in the filtrate was removed by a rotary evaporator and dried under reduced pressure (120° C., 8 hr) to give compound (1) in a yield of not less than 95%.

The results of the infrared spectrum as measured of the obtained compound (1) are shown in the following:
disappearance of peak at around 640 cm⁻¹, which was derived from C—Br in the starting material,
appearance of peak at around 1250 cm⁻¹, which was derived from a phenol ether bond and
slight decrease in the peak intensity at 3200 cm⁻¹, which was derived from a hydroxy group.

Based on the yield and infrared spectrum above, compound (1) can be said to mainly have a structure represented by the above-mentioned formula (1), wherein, in the above-mentioned formula (1), R₁ is a hydrogen atom, R₂ is a tert-butyl group, R₃ is a hydrogen atom, R₄ is a stearyl group, m is 2, n is approximately 0, 1 is about 6, and n+m+1 is 8.

### Example 2

Synthesis of Compound (2)

A 60% sodium hydride-oil dispersion (0.43 part, manufactured by Wako Pure Chemical Industries, Ltd.) was collected in a flask, oil was washed with THF (manufactured by JUNSEI CHEMICAL CO., LTD.), THF (8.87 parts) was added as a reaction solvent into the flask, and a mixer and a distillation column were set. Thereto was added, over 10 min with stirring, 4-tert-butylcalix(8)arene (7.00 parts, manufactured by Kawaguchi Chemical Co., Ltd.) dispersed in advance in THF (22.18 parts) in a beaker, and the beaker was washed with THF (13.31 parts) and refluxed for 3 hr.

11-Bromo-1-undecanol (3.20 parts, 2.25-fold mol relative to 4-tert-butylcalix(8)arene, manufactured by ACROS ORGANICS) was dissolved in THF (13.31 parts) in advance, and collected in a dropping funnel, set on a flask containing a reaction product of the above-mentioned 4-tert-butylcalix(8)arene and sodium hydride together with a stirrer and a distillation column, and a 11-bromo-1-undecanol/THF solution was added dropwise over 15 min with stirring. After the completion of the dropwise addition, the dropping funnel was washed with THF (4.44 parts), which was followed by refluxing for 3 hr.

Using a Kiriyama funnel and a paper filter (No. 5C), excess sodium hydride in the above-mentioned reaction product was removed, and the filtrate was neutralized with acetic acid (manufactured by JUNSEI CHEMICAL CO., LTD.). Then, the precipitate was removed with a Kiriyama funnel and a paper filter (No. 5C), and the filtrate was passed through a syringe filter (0.2 μm) to further remove fine precipitate. The solvent in the filtrate was removed by a rotary evaporator and dried under reduced pressure (120° C., 8 hr) to give compound (2) in a yield of not less than 95%.

The results of the infrared spectrum as measured of the obtained compound (2) are shown in the following:
disappearance of peak at 640 cm⁻¹, which was derived from C—Br in the starting material,
appearance of peak at around 1250 cm⁻¹, which was derived from a henol ether bond and
slight decrease in the peak intensity at 3200 cm⁻¹, which was derived from a hydroxy group.

Based on the yield and infrared spectrum above, compound (2) can be said to mainly have a structure represented by the above-mentioned formula (1), wherein R₁ is a hydrogen atom, R₂ is a tert-butyl group, R₃ is a hydrogen atom, R₄ is a 11-hydroxyundecyl group, m is 2.25, n is approximately 0, 1 is about 5.75, and n+m+1 is 8.

### Example 3

Synthesis of Compound (3)

4-tert-Butylcalix(8)arene (5.00 parts, manufactured by Kawaguchi Chemical Co., Ltd.), triethylamine (3.10 parts, manufactured by JUNSEI CHEMICAL CO., LTD.) and acetone (100 parts, manufactured by JUNSEI CHEMICAL CO., LTD.) were placed in a beaker, and the mixture was stirred at room temperature for 1 hr to give an almost transparent mixture. This was filtered with a Kiriyama funnel and a paper filter (No. 5C) to give a transparent solution. This solution was placed in a flask, a solution of stearic acid chloride (2.33 parts, 2-fold mol relative to 4-tert-butylcalix(8)arene, manufactured by Tokyo Kasei Kogyo Co., Ltd.) in acetone (10.0 parts, manufactured by JUNSEI CHEMICAL CO., LTD.) was added dropwise, and the mixture was stirred at room temperature for 4 hr. Acetic acid (1.38 parts, manufactured by JUNSEI CHEMICAL CO., LTD.) was added thereto, and the mixture was stirred at room temperature for 4 hr. Then the mixture was filtered with a Kiriyama funnel and a paper filter (No. 5C), the resulting salts were removed, and the solvent was evaporated by a rotary evaporator to give a white solid. This was re-dissolved in toluene (manufactured by JUNSEI CHEMICAL CO., LTD.) and placed in a separatory funnel, water was added to partition the solution, and the toluene layer was separated and dried over anhydrous magnesium sulfate. Toluene was evaporated by a rotary evaporator, and the residue was dried under reduced pressure at 110° C. to give compound (3) in a yield of not less than 95%.

The results of the infrared spectrum as measured of the obtained compound (3) are shown in the following:
disappearance of peak at around 1780 cm⁻¹, which was derived from an acid chloride (starting material), appearance of peak at around 1720 cm⁻¹, which was derived from a phenol ether bond and slight decrease in the peak intensity at 3200 cm⁻¹, which was derived from a hydroxy group.

Based on the yield and infrared spectrum above, compound (3) can be said to mainly have a structure represented by the above-mentioned formula (1), wherein R₁ is a hydrogen atom, R₂ is a tert-butyl group, R₃ is a hydrogen atom, R₄ is a stearoyl group, m is 2, n is approximately 0, 1 is about 6, and n+m+1 is 8.

### Example 4

Synthesis of Compound (4)

4-tert-Butylcalix(8)arene (5.00 parts, manufactured by Kawaguchi Chemical Co., Ltd.), triethylamine (3.10 parts, manufactured by JUNSEI CHEMICAL CO., LTD.) and acetone (100 parts, manufactured by JUNSEI CHEMICAL CO., LTD.) were placed in a beaker, and the mixture was stirred at room temperature for 1 hr to give an almost transparent mixture. This was filtered with a Kiriyama funnel and a paper filter (No. 5C) to give a transparent solution. This solution was placed in a flask, a solution of decanoyl chloride (1.47 parts, 2-fold mol relative to 4-tert-butylcalix(8)arene, manufactured by JUNSEI CHEMICAL CO., LTD.) in acetone (10.0 parts, manufactured by JUNSEI CHEMICAL CO., LTD.) was added dropwise, and the mixture was stirred at room temperature for 4 hr. Acetic acid (1.38 parts, manufactured by JUNSEI CHEMICAL CO., LTD.) was added thereto, and the mixture was stirred at room temperature for 4 hr. Then the mixture was filtered with a Kiriyama funnel and a paper filter (No. 5C), the resulting salts were removed, and the solvent was evaporated by a rotary evaporator to give a white solid. This was re-dissolved in toluene (manufactured by JUNSEI CHEMICAL CO., LTD.) and placed in a separatory funnel, water was added to partition the solution, and the toluene layer was separated and dried over anhydrous magnesium sulfate Toluene was evaporated by a rotary evaporator, and the residue was dried under reduced pressure at 110° C. to give compound (4) in a yield of not less than 95%.

The results of the infrared spectrum as measured of the obtained compound (4) are shown in the following:
disappearance of peak at around 1780 cm⁻¹, which was derived from an acid chloride (starting material),
appearance of peak at around 1720 cm⁻¹, which was derived from a phenol ether bond and
slight decrease in the peak intensity at 3200 cm⁻¹, which was derived from a hydroxy group.

Based on the yield and infrared spectrum above, compound (4) can be said to mainly have a structure represented by the above-mentioned formula (1), wherein R₁ is a hydrogen atom, R₂ is a tert-butyl group, R₃ is a hydrogen atom, R₄ is a decanoyl group, m is 2, n is approximately 0, 1 is about 6, and n+m+1 is 8.

### Example 5

Synthesis of Compound (5)

4-tert-Butylcalix(8)arene (5.00 parts, manufactured by Kawaguchi Chemical Co., Ltd.), triethylamine (3.10 parts, manufactured by JUNSEI CHEMICAL CO., LTD.) and acetone (100 parts, manufactured by JUNSEI CHEMICAL CO., LTD.) were placed in a beaker, and the mixture was stirred at room temperature for 1 hr to give an almost transparent mixture. This was filtered with a Kiriyama funnel and a paper filter (No. 5C) to give a transparent solution. This solution was placed in a flask, a solution of stearic acid chloride (4.66 parts, 4-fold mol relative to 4-tert-butylcalix(8)arene, manufactured by JUNSEI CHEMICAL CO., LTD.) in acetone (10.0 parts, manufactured by JUNSEI CHEMICAL CO., LTD.) was added dropwise, and the mixture was stirred at room temperature for 16 hr. Acetic acid (0.92 part, manufactured by JUNSEI CHEMICAL CO., LTD.) was added thereto, and the mixture was stirred at room temperature for 4 hr. Then the mixture was filtered with a Kiriyama funnel and a paper filter (No. 5C), the resulting salts were removed, and the solvent was evaporated by a rotary evaporator to give a white solid. This was re-dissolved in a 1:1 mixed solvent of toluene (manufactured by JUNSEI CHEMICAL CO., LTD.) and hexane (manufactured by JUNSEI CHEMICAL CO., LTD.) and placed in a separatory funnel, water was added to partition the solution, the toluene-hexane layer was separated and dried over anhydrous magnesium sulfate, toluene and hexane were evaporated by a rotary evaporator, and the residue was dried under reduced pressure at 110° C. to give compound (5) in a yield of not less than 95%.

The results of the infrared spectrum as measured of the obtained compound (5) are shown in the following:
disappearance of peak at around 1780 cm⁻¹, which was derived from an acid chloride (starting material),
appearance of peak at around 1720 cm⁻¹, which was derived from a phenol ether bond and
decrease to half in the intensity of the peak intensity at 3200 cm⁻¹, which was derived from a hydroxy group.

Based on the yield and infrared spectrum above, compound (5) can be said to mainly have a structure represented by the above-mentioned formula (1), wherein R₁ is a hydrogen atom, R₂ is a tert-butyl group, R₃ is a hydrogen atom, R₄ is a stearoyl group, m is 4, n is approximately 0, 1 is about 4, and n+m+1 is 8.

### Example 6

Synthesis of Compound (6)

4-tert-Butylcalix(8)arene (5.00 parts, manufactured by Kawaguchi Chemical Co., Ltd.), triethylamine (3.10 parts, manufactured by JUNSEI CHEMICAL CO., LTD.) and acetone (100 parts, manufactured by JUNSEI CHEMICAL CO., LTD.) were placed in a beaker, and the mixture was stirred at room temperature for 1 hr to give an almost transparent mixture. This was filtered with a Kiriyama funnel and a paper filter (No. 5C) to give a transparent solution. This solution was placed in a flask, a solution of stearic acid chloride (6.99 parts, 6-fold mol relative to 4-tert-butylcalix(8)arene, manufactured by Tokyo Kasei Kogyo Co., Ltd.) in acetone (10.0 parts, manufactured by JUNSEI CHEMICAL CO., LTD.) was added dropwise, and the mixture was stirred at room temperature for 16 hr. Acetic acid (0.46 part, manufactured by JUNSEI CHEMICAL CO., LTD.) was added thereto, and the mixture was stirred at room temperature for 4 hr. Then the mixture was filtered with a Kiriyama funnel and a paper filter (No. 5C), the resulting salts were removed, and the solvent was evaporated by a rotary evaporator to give a white solid. This was dissolved in hexane (manufactured by JUNSEI CHEMICAL CO., LTD.) and placed in a separatory funnel, water was added to partition the solution, the hexane layer was separated and dried over anhydrous magnesium sulfate, hexane was evaporated by a rotary evaporator, and the residue was dried under reduced pressure at 110° C. to give compound (6) in a yield of not less than 95%.

The results of the infrared spectrum as measured of the obtained compound (6) are shown in the following:
disappearance of peak at around 1780 cm⁻¹, which was derived from an acid chloride (starting material),
appearance of peak at around 1720 cm⁻¹, which was derived from a phenol ether bond and
decrease in the peak intensity at 3200 cm⁻¹, which was derived from a hydroxy group.

Based on the yield and infrared spectrum above, compound (6) can be said to mainly have a structure represented by the above-mentioned formula (1), wherein R₁ is a hydrogen atom, R₂ is a tert-butyl group, R₃ is a hydrogen atom, R₄ is a stearoyl group, m is 6, n is approximately 0, 1 is about 2, and n+m+1 is 8.

### Example 7

Synthesis of Compound (7)

4-tert-Butylcalix(8)arene (5.00 parts, manufactured by Kawaguchi Chemical Co., Ltd.), triethylamine (3.10 parts, manufactured by JUNSEI CHEMICAL CO., LTD.) and acetone (100 parts, manufactured by JUNSEI CHEMICAL CO., LTD.) were placed in a beaker, and the mixture was stirred at room temperature for 1 hr to give an almost transparent mixture. This was filtered with a Kiriyama funnel and a paper filter (No. 5C) to give a transparent solution. This solution was placed in a flask, a solution of adipic acid dichloride (0.353 part, 0.5-fold mol relative to 4-tert-butylcalix(8)arene, manufactured by JUNSEI CHEMICAL CO., LTD. and 2-fold mol relative to stearic acid chloride (2.33 parts, 4-tert-butylcalix(8)arene)) in acetone (15.0 parts, manufactured by JUNSEI CHEMICAL CO., LTD.) was added dropwise, and the mixture was stirred at room temperature for 4 hr. Acetic acid (0.92 part, manufactured by JUNSEI CHEMICAL CO., LTD.) was added thereto, and the mixture was stirred at room temperature for 4 hr. Then the mixture was filtered with a Kiriyama funnel and a paper filter (No. 5C), the resulting salts were removed, and the solvent was evaporated by a rotary evaporator to give a white solid. This was dissolved in hexane (manufactured by JUNSEI CHEMICAL CO., LTD.) and placed in a separatory funnel, water was added to partition the solution, the hexane layer was separated and dried over anhydrous magnesium sulfate, hexane was evaporated by a rotary evaporator, and the residue was dried under reduced pressure at 110° C. to give compound (7) in a yield of not less than 95%.

The results of the infrared spectrum as measured of the obtained compound (7) are shown in the following:
disappearance of peak at around 1780 cm⁻¹, which was derived from an acid chloride (starting material),
appearance of peak at around 1720 cm⁻¹, which was derived from a phenol ether bond and
decrease in the peak intensity at 3200 cm⁻¹, which was derived from a hydroxy group.

Based on the yield and infrared spectrum above, compound (7) can be said to mainly have a structure represented by the above-mentioned formula (2), wherein R₁′ is a hydrogen atom, R₂′ is a tert-butyl group, R₃′ is a hydrogen atom, R₄′ is a stearoyl group, R₅ is a tetramethylene group, q is 1, s and s′ are each 2, p and p′ are each approximately 0, r and r′ are each about 5, and p+q+r+s and p′+q+r′+s′ are each 8.

### Example 8

Synthesis of Compound (8)

Phosphorus tribromide (0.09 part, manufactured by Wako Pure Chemical Industries, Ltd.) was collected in a flask with a stirring bar. Polyethylene glycol monostearate (3.38 parts, 2-fold mol relative to 4-tert-butylcalix(8)arene, average degree of polymerization about 45, molecular weight about 2200, manufactured by Tokyo Kasei Kogyo Co., Ltd.) was collected in a dropping funnel, which was set on the flask with a calcium chloride tube, and added dropwise over 30 min in an ice bath with stirring. After the completion of the dropwise addition, the mixture was stirred in an ice bath for 2 hr and allowed to return to room temperature over 1 hr with further stirring, and the mixture was stirred at room temperature for 24 hr. Then, THF (20 parts, manufactured by JUNSEI CHEMICAL CO., LTD.) was added to the flask in several portions, the mixture was stirred, and the solvent was taken out into the dropping funnel, which step was repeated to give components soluble in THF in the dropping funnel.

A 60% sodium hydride-oil dispersion (0.62 part, manufactured by Wako Pure Chemical Industries, Ltd.) was collected in a different flask, and the oil was washed with THF (manufactured by JUNSEI CHEMICAL CO., LTD.). THF (8.87 parts) was added as a reaction solvent into the flask, and a mixer and a distillation column were set. Thereto was added, over 15 min with stirring, 4-tert-butylcalix(8)arene (1.00 parts, manufactured by Kawaguchi Chemical Co., Ltd.) dispersed in THF (13.31 parts) in advance in a beaker, and the beaker was washed with THF (4.44 parts) and refluxed for 3 hr. After the completion of refluxing, the mixture was allowed to cool, excess sodium hydride in the above-mentioned reaction product was removed using a Kiriyama funnel and a paper filter (No. 5C), and THF (17.74 parts) was added as a reaction solvent into the flask of the filtrate.

A dropping funnel containing the reaction product of the aforementioned polyethylene glycol monostearate and phosphorus tribromide was set on a pear shape flask containing a reaction product of the above-mentioned 4-tert-butylcalix(8)arene and sodium hydride, and the product was added dropwise over 30 min at room temperature. Thereafter, and the mixture was refluxed in a mixed solvent for 3 hr.

After the completion of refluxing, the mixture was allowed to cool and was neutralized by adding acetic acid (manufactured by JUNSEI CHEMICAL CO., LTD.) into the flask. Then, the precipitate was removed with a Kiriyama funnel and a paper filter (No. 5C). The solvent in the filtrate was removed by a rotary evaporator to give compound (8) in a yield of not less than 95%.

The results of the infrared spectrum as measured of the obtained compound (8) are shown in the following:
disappearance of peak at around 640 cm⁻¹, which was derived from C—Br in the intermediate product,
appearance of peak at around 1250 cm⁻¹, which was derived from a phenol ether bond and
slight decrease in the peak intensity at 3200 cm⁻¹, which was derived from a hydroxy group.

Based on the yield and infrared spectrum above, compound (8) can be said to mainly have a structure represented by the above-mentioned formula (1), wherein R₁ is a hydrogen atom, R₂ is a tert-butyl group, R₃ is a hydrogen atom, R₄ is a terminally stearoylated polyethylene glycol chain, m is 2, n is approximately 0, 1 is about 6, and n+m+1 is 8.

### Example 9

Synthesis of Compound (9)

Stearyl alcohol (10.00 parts, manufactured by JUNSEI CHEMICAL CO., LTD.), ε-caprolactone (60.0 parts, manufactured by JUNSEI CHEMICAL CO., LTD.) and tetrabutyl titanate (0.01 part, manufactured by JUNSEI CHEMICAL CO., LTD.) were collected in a flask with a stirring bar, and the mixture was heated to 160° C. over 4 hr. The mixture was heated at 160° C. for 2 hr to give a terminal stearyl ester of caprolactone polymer (degree of polymerization 26, number average molecular weight 2604).

Phosphorus tribromide (0.09 part, manufactured by Wako Pure Chemical Industries, Ltd.) was collected in a flask with a stirring bar, and the above-mentioned caprolactone polymer (4.01 parts, 2-fold mol relative to 4-tert-butylcalix(8)arene) was collected in a dropping funnel, which was set on the flask with a calcium chloride tube, and added dropwise over 30 min in an ice bath with stirring. After the completion of the dropwise addition, the mixture was stirred in an ice bath for 2 hr, allowed to return to room temperature over 1 hr with further stirring, and then the mixture was stirred at room temperature for 24 hr. Then, THF (20 parts, manufactured by JUNSEI CHEMICAL CO., LTD.) was added to the flask in several portions, the mixture was stirred, and the solvent was taken out into the dropping funnel, which step was repeated to give components soluble in THF in the dropping funnel.

A 60% sodium hydride-oil dispersion (0.62 part, manufactured by Wako Pure Chemical Industries, Ltd.) was collected in a different flask, and the oil was washed with THF (manufactured by JUNSEI CHEMICAL CO., LTD.). THF (8.87 parts) was added as a reaction solvent into the flask, and a mixer and a distillation column were set. Thereto was added, over 15 min with stirring, 4-tert-butylcalix(8)arene (1.00 parts, manufactured by Kawaguchi Chemical Co., Ltd.) dispersed in THF (13.31 parts) in advance in a beaker, and the beaker was washed with THF (4.44 parts) and refluxed for 3 hr. After the completion of refluxing, the mixture was allowed to cool, excess sodium hydride in the above-mentioned reaction product was removed using a Kiriyama funnel and a paper filter (No. 5C), and THF (17.74 parts) was added as a reaction solvent into the flask of the filtrate.

A dropping funnel containing the reaction product of the aforementioned caprolactone polymer and phosphorus tribromide was set on a pear shape flask containing a reaction product of the above-mentioned 4-tert-butylcalix(8)arene and sodium hydride, and the product was added dropwise over 30 min at room temperature. Thereafter, and the mixture was refluxed in a mixed solvent for 3 hr.

After the completion of refluxing, the mixture was allowed to cool and neutralized by adding acetic acid (manufactured by JUNSEI CHEMICAL CO., LTD.) into the flask. Then, the precipitate was removed using a Kiriyama funnel and a paper filter (No. 5C). The solvent in the filtrate was removed by a rotary evaporator to give compound (9) in a yield of not less than 95%.

The results of the infrared spectrum as measured of the obtained compound (9) are shown in the following:
disappearance of peak at around 640 cm⁻¹, which was derived from C—Br in the intermediate product,
appearance of peak at around 1250 cm⁻¹, which was derived from a phenol ether bond and
slight decrease in the peak intensity at 3200 cm⁻¹, which was derived from a hydroxy group.

Based on the yield and infrared spectrum above, compound (9) can be said to mainly have a structure represented by the above-mentioned formula (1), wherein R₁ is a hydrogen atom, R₂ is a tert-butyl group, R₃ is a hydrogen atom, R₄ is a terminally stearylated caprolactone polymer chain, m is 2, n is approximately 0, 1 is about 6, and n+m+1 is 8.

### Example 10

A hydroxyl group-terminally stearoylated caprolactone polymer (13.72 parts (2-fold mol relative to t-butylcalix(8)arene), manufactured by Kawaken Fine Chemicals Co., Ltd., molecular weight about 3,000) was dissolved in toluene (51.90 parts, manufactured by JUNSEI CHEMICAL CO., LTD.) in a pear shape flask. Thereto was added dropwise thionyl chloride (1.65 parts, manufactured by JUNSEI CHEMICAL CO., LTD.) at room temperature over about 10 min and, after the completion of the dropwise addition, the dropping funnel was washed with toluene (2.60 parts, manufactured by JUNSEI CHEMICAL CO., LTD.), and the mixture was stirred one day at room temperature. t-Butylcalix(8)arene (3.00 parts, manufactured by Kawaguchi Chemical Co., Ltd.) and acetone (28.48 parts, manufactured by JUNSEI CHEMICAL CO., LTD.) were added to a different pear shape flask, and the mixture was stirred at room temperature to allow t-butylcalix(8)arene to disperse in the acetone. To the dispersion was added triethylamine (1.89 parts, manufactured by JUNSEI CHEMICAL CO., LTD.), and the mixture was stirred to give a transparent acetone solution. The reaction product of the hydroxyl group-terminally stearoylated caprolactone polymer (manufactured by Kawaken Fine Chemicals Co., Ltd.) and thionyl chloride (manufactured by JUNSEI CHEMICAL CO., LTD.) reacted earlier was collected as the reaction mixture in a dropping funnel and quickly added dropwise to the obtained acetone solution. After the completion of the dropwise addition, the dropping funnel was washed with acetone (2.37 parts, manufactured by JUNSEI CHEMICAL CO., LTD.), and the mixture was stirred at room temperature for 5 hr. Acetic acid (0.83 part (6-fold mol relative to t-butylcalixarene, manufactured by JUNSEI CHEMICAL CO., LTD.) was added, and the mixture was stirred at room temperature for 1 hr to perform neutralization. Thereto was added pure water, and the mixture was stirred at room temperature for 1 hr to quench unreacted thionyl chloride. Then, the solvent was evaporated by a rotary evaporator to give a white solid. This was re-dissolved in toluene (manufactured by JUNSEI CHEMICAL CO., LTD.) and placed in a separatory funnel, and water was added to allow for partitioning. The toluene layer was separated and dried over anhydrous magnesium sulfate. Toluene was evaporated by a rotary evaporator and dried under reduced pressure at 110° C. to give compound (10) in a yield of 93%.

The results of the infrared spectrum as measured of the obtained compound (10) are shown in the following:
disappearance of peak at around 1780 cm⁻¹, which was derived from an acid chloride in the intermediate product,
appearance of peak at around 1720 cm⁻¹, which was derived from a phenol ether bond and
decrease in the peak intensity at 3200 cm⁻¹, which was derived from a hydroxy group.

Based on the yield and infrared spectrum above, compound (10) can be said to mainly have a structure represented by the above-mentioned formula (1), wherein R₁ is a hydrogen atom, R₂ is a tert-butyl group, R₃ is a hydrogen atom, R₄ is a hydroxyl group-terminally stearoylated caprolactone polymer chain, m is 2, n is approximately 0, 1 is 6, and n+m+1 is 8.

### Examples 11-20

The compounds (1)-(10) (each 20.0 parts) obtained in Examples 1-10 were dissolved in toluene (200 parts). A solution of C₆₀ (0.2 part, manufactured by Tokyo Kasei Kogyo Co., Ltd.) dissolved in toluene (200 parts) was added to each of these solutions and, after stirring at room temperature for 20 min, solvent was removed by a rotary evaporator to give fullerene composites (1)-(10).

Hexane (200 parts) was added to these fullerene composites (1)-(10). As a result, they became transparent solutions free of insoluble material, and no precipitate was observed even after 24 hr.

### Comparative Example 1

A 60% sodium hydride-oil dispersion (0.31 part, manufactured by Wako Pure Chemical Industries, Ltd.) was collected in a flask, and the oil was washed with THF (manufactured by JUNSEI CHEMICAL CO., LTD.). THF (6.33 parts) was added as a reaction solvent into the flask, and a mixer and a distillation column were set. Thereto was added, over 10 min with stirring, 4-tert-butylcalix(8)arene (5.00 parts, manufactured by Kawaguchi Chemical Co., Ltd.) dispersed in THF (15.8 parts) in a beaker, and the beaker was washed with THF (9.5 parts) and refluxed for 3 hr.

Stearic acid chloride (9.32 parts, 8-fold mol relative to 4-tert-butylcalix(8)arene, manufactured by JUNSEI CHEMICAL CO., LTD.) was dissolved in THF (10 parts) in advance and collected in a dropping funnel, which was set, together with a mixer and a distillation column, on the flask containing the reaction product of the above-mentioned 4-tert-butylcalix(8)arene and sodium hydride, and the mixture was added dropwise over 15 min with stirring. After the completion of the dropwise addition, the dropping funnel was washed with THF (5 parts) and the mixture was refluxed for 3 hr.

Excess sodium hydride in the above-mentioned reaction product was removed using a Kiriyama funnel and a paper filter (No. 5C), and acetic acid (0.1 part, manufactured by JUNSEI CHEMICAL CO., LTD.) was added to the filtrate to perform neutralization. Then, the precipitate was removed with a Kiriyama funnel and a paper filter (No. 5C). The solvent in the filtrate was removed by a rotary evaporator, then the residue was re-dissolved in hexane (manufactured by JUNSEI CHEMICAL CO., LTD.) and placed in a separatory funnel. Water was added to perform partitioning, and the hexane layer was separated over anhydrous magnesium sulfate. Hexane was evaporated with a rotary evaporator and dried under reduced pressure at 110° C. to give comparison compound (1) in a yield of not less than 95%.

The results of the infrared spectrum as measured of the obtained comparison compound (1) are shown in the following:
disappearance of peak at around 1780 cm⁻¹, which was derived from an acid chloride (starting material),
appearance of peak at around 1720 cm⁻¹, which was derived from a phenol ether bond and
disappearance of peak at 3200 cm⁻¹, which was derived from a hydroxy group.

Based on the yield and infrared spectrum above, comparison compound (1) can be said to mainly have a structure represented by the above-mentioned formula (1), wherein R₁ is a hydrogen atom, R₂ is a tert-butyl group, R₃ is a hydrogen atom, R₄ is a stearoyl group, m is 8, n and 1 is approximately 0, and n+m+1 is 8.

### Comparative Example 2

### Synthesis of Comparison Compound (2)

4-tert-Butylcalix(8)arene (5.00 parts, manufactured by Kawaguchi Chemical Co., Ltd.), triethylamine(3.10 parts, manufactured by JUNSEI CHEMICAL CO., LTD.) and acetone (100 parts, manufactured by JUNSEI CHEMICAL CO., LTD.) were placed in a beaker, and the mixture was stirred at room temperature for 1 hr to give an almost transparent mixture. This was filtered using a Kiriyama funnel and a paper filter (No. 5C) to give a transparent solution. This solution was placed in a flask, a solution of butyryl chloride (0.82 part, 2-fold mol relative to 4-tert-butylcalix(8)arene, manufactured by Tokyo Kasei Kogyo Co., Ltd.) in acetone (10.0 parts, manufactured by JUNSEI CHEMICAL CO., LTD.) was added dropwise, and the mixture was stirred at room temperature for 4 hr. Acetic acid (1.38 parts, manufactured by JUNSEI CHEMICAL CO., LTD.) was added thereto, and the mixture was stirred at room temperature for 4 hr. Then the mixture was filtered with a Kiriyama funnel and a paper filter (No. 5C), the resulting salts were removed, and the solvent was evaporated by a rotary evaporator to give a white solid. This was dissolved in toluene (manufactured by JUNSEI CHEMICAL CO., LTD.) and placed in a separatory funnel, and water was added to partition the solution. The toluene layer was separated and dried over anhydrous magnesium sulfate. Toluene was evaporated by a rotary evaporator and dried under reduced pressure at 110° C. to give comparison compound (2) in a yield of not less than 95%.

The results of the infrared spectrum as measured of the obtained comparison compound (2) are shown in the following:
disappearance of peak at around 1780 cm⁻¹, which was derived from an acid chloride (starting material),
appearance of peak at around 1720 cm⁻¹, which was derived from a phenol ether bond and
slight decrease in the peak intensity at 3200 cm⁻¹, which was derived from a hydroxy group.

Based on the yield and infrared spectrum above, comparison compound (2) can be said to mainly have a structure represented by the above-mentioned formula (1), wherein R₁ is a hydrogen atom, R₂ is a tert-butyl group, R₃ is a hydrogen atom, R₄ is a butanoyl group, m is 2, n is approximately 0, 1 is about 6, and n+m+1 is 8.

### Comparative Example 3

### Synthesis of Comparison Compound (3)

4-tert-Butylcalix(8)arene (5.00 parts, manufactured by Kawaguchi Chemical Co., Ltd.), triethylamine (3.10 parts, manufactured by JUNSEI CHEMICAL CO., LTD.) and acetone (100 parts, manufactured by JUNSEI CHEMICAL CO., LTD.) were placed in a beaker, and the mixture was stirred at room temperature for 1 hr to give an almost transparent mixture. This was filtered using a Kiriyama funnel and a paper filter (No. 5C) to give a transparent solution. This solution was placed in a flask, a solution of butyryl chloride (1.64 parts, 4-tert-butylcalix(8)arene 4-fold mol relative to, manufactured by Tokyo Kasei Kogyo Co., Ltd.) in acetone (10.0 parts, manufactured by JUNSEI CHEMICAL CO., LTD.) was added dropwise, and the mixture was stirred at room temperature for 4 hr. Acetic acid (0.92 part, manufactured by JUNSEI CHEMICAL CO., LTD.) was added thereto, and the mixture was stirred at room temperature for 4 hr. Then the mixture was filtered with a Kiriyama funnel and a paper filter (No. 5C), the resulting salts were removed, and the solvent was evaporated by a rotary evaporator to give a white solid. This was dissolved in toluene (manufactured by JUNSEI CHEMICAL CO., LTD.) and placed in a separatory funnel, and water was added to partition the solution. The toluene layer was separated and dried over anhydrous magnesium sulfate. Toluene was evaporated by a rotary evaporator and dried under reduced pressure at 110° C. to give comparison compound (3) in a yield of not less than 95%.

The results of the infrared spectrum as measured of the obtained comparison compound (3) are shown in the following:
disappearance of peak at around 1780 cm⁻¹, which was derived from an acid chloride (starting material),
appearance of peak at around 1720 cm⁻¹, which was derived from a phenol ether bond and
decrease to half in the peak intensity at 3200 cm⁻¹, which was derived from a hydroxy group.

Based on the yield and infrared spectrum above, comparison compound (3) can be said to mainly have a structure represented by the above-mentioned formula (1), wherein R₁ is a hydrogen atom, R₂ is a tert-butyl group, R₃ is a hydrogen atom, R₄ is a butanoyl group, m is 4, n is approximately 0, 1 is about 4, and n+m+1 is 8.

### Comparative Example 4

### Synthesis of Comparison Compound (4)

4-tert-Butylcalix(8)arene (5.00 parts, manufactured by Kawaguchi Chemical Co., Ltd.), triethylamine (3.10 parts, manufactured by JUNSEI CHEMICAL CO., LTD.) and acetone (100 parts, manufactured by JUNSEI CHEMICAL CO., LTD.) were placed in a beaker, and the mixture was stirred at room temperature for 1 hr to give an almost transparent mixture. This was filtered using a Kiriyama funnel and a paper filter (No. 5C) to give a transparent solution. This solution was placed in a flask, a solution of hexanoyl chloride (1.03 parts, 2-fold mol relative to 4-tert-butylcalix(8)arene, manufactured by Tokyo Kasei Kogyo Co., Ltd.) in acetone (10.0 parts, manufactured by JUNSEI CHEMICAL CO., LTD.) was added dropwise, and the mixture was stirred at room temperature for 4 hr. Acetic acid (1.38 parts, manufactured by JUNSEI CHEMICAL CO., LTD.) was added thereto, and the mixture was stirred at room temperature for 4 hr. Then the mixture was filtered with a Kiriyama funnel and a paper filter (No. 5C), the resulting salts were removed, and the solvent was evaporated by a rotary evaporator to give a white solid. This was dissolved in toluene (manufactured by JUNSEI CHEMICAL CO., LTD.) and placed in a separatory funnel, and water was added to partition the solution. The toluene layer was separated and dried over anhydrous magnesium sulfate. Toluene was evaporated by a rotary evaporator and dried under reduced pressure at 110° C. to give comparison compound (4) in a yield of not less than 95%.

The results of the infrared spectrum as measured of the obtained comparison compound (4) are shown in the following:
disappearance of peak at around 1780 cm⁻¹, which was derived from an acid chloride (starting material),
appearance of peak at around 1720 cm⁻¹, which was derived from a phenol ether bond and
slight decrease in the peak intensity at 3200 cm⁻¹, which was derived from a hydroxy group.

Based on the yield and infrared spectrum above, comparison compound (4) can be said to mainly have a structure represented by the above-mentioned formula (1), wherein R₁ is a hydrogen atom, R₂ is a tert-butyl group, R₃ is a hydrogen atom, R₄ is a hexanoyl group, m is 2, n is approximately 0, 1 is about 6, and n+m+1 is 8.

### Comparative Example 5

### Synthesis of Comparison Compound (5)

4-tert-Butylcalix(8)arene (5.00 parts, manufactured by Kawaguchi Chemical Co., Ltd.), triethylamine (3.10 parts, manufactured by JUNSEI CHEMICAL CO., LTD.) and acetone (100 parts, manufactured by JUNSEI CHEMICAL CO., LTD.) were placed in a beaker, and the mixture was stirred at room temperature for 1 hr to give an almost transparent mixture. This was filtered using a Kiriyama funnel and a paper filter (No. 5C) to give a transparent solution. This solution was placed in a flask, a solution of octanoyl chloride (1.25 parts, 2-fold mol relative to 4-tert-butylcalix(8)arene, manufactured by Tokyo Kasei Kogyo Co., Ltd.) in acetone (10.0 parts, manufactured by JUNSEI CHEMICAL CO., LTD.) was added dropwise, and the mixture was stirred at room temperature for 4 hr. Acetic acid (1.38 parts, manufactured by JUNSEI CHEMICAL CO., LTD.) was added thereto, and the mixture was stirred at room temperature for 4 hr. Then the mixture was filtered with a Kiriyama funnel and a paper filter (No. 5C), the resulting salts were removed, and the solvent was evaporated by a rotary evaporator to give a white solid. This was dissolved in toluene (manufactured by JUNSEI CHEMICAL CO., LTD.) and placed in a separatory funnel, and water was added to partition the solution. The toluene layer was separated and dried over anhydrous magnesium sulfate. Toluene was evaporated by a rotary evaporator and dried under reduced pressure at 110° C. to give comparison compound (5) in a yield of not less than 95%.

The results of the infrared spectrum as measured of the obtained comparison compound (5) are shown in the following:
disappearance of peak at around 1780 cm⁻¹, which was derived from an acid chloride (starting material),
appearance of peak at around 1720 cm⁻¹, which was derived from a phenol ether bond and
slight disappearance of the peak intensity at 3200 cm⁻¹, which was derived from a hydroxy group.

Based on the yield and infrared spectrum above, comparison compound (5) can be said to mainly have a structure represented by the above-mentioned formula (1), wherein R₁ is a hydrogen atom, R₂ is a tert-butyl group, R₃ is a hydrogen atom, R₄ is a octanoyl group, m is 2, n is approximately 0, 1 is about 6, and n+m+1 is 8.

### Comparative Example 6

C₆₀ (0.2 part, manufactured by Tokyo Kasei Kogyo Co., Ltd.) was added to hexane (200 parts) but it was scarcely dissolved, and a black insoluble material was precipitated.

### Comparative Examples 7-11

The comparison compounds (1)-(5) (each 20.0 parts) obtained in Comparative Examples 1-5 were dissolved in toluene (200 parts). A solution of C₆₀ (0.2 part, manufactured by Tokyo Kasei Kogyo Co., Ltd.) dissolved in toluene (200 parts) was added to each of these solutions, and, after stirring at room temperature for 20 min, solvent was removed by a rotary evaporator to give comparison fullerene composites (1)-(5).

Hexane (200 parts) was added to each of these comparison fullerene composites (1)-(5), and the mixture was stirred. As a result, comparison fullerene composites (1)-(4) showed a remaining black insoluble material and did not dissolve completely. After 24 hr, the precipitate increased. While comparison fullerene composite (5) was dissolved, a precipitate occurred 24 hr later.

### Comparative Example 12

A titanate coupling agent (20.0 parts, manufactured by Ajinomoto-Fine-Techno Co., Inc., Plenact KR TTS) was dissolved in toluene (200 parts). A solution obtained by dissolving C₆₀ (0.2 part, manufactured by Tokyo Kasei Kogyo Co., Ltd.) in toluene (200 parts) was added to this solution, and the mixture was stirred at room temperature for 20 min. The solvent was removed by a rotary evaporator to give comparison fullerene composite (6).

Hexane (200 parts) was added to this comparison fullerene composite (6), and the mixture was stirred. As a result, the comparison fullerene composite was scarcely dissolved, and a black insoluble material was precipitated.

### Comparative Example 13

An aluminate-based coupling agent (20.0 parts, manufactured by Ajinomoto-Fine-Techno Co., Inc., Plenact AL-M) was dissolved in toluene (200 parts). A solution obtained by dissolving C₆₀ (0.2 part, manufactured by Tokyo Kasei Kogyo Co., Ltd.) in toluene (200 parts) was added to this solution, and the mixture was stirred at room temperature for 20 min. The solvent was removed by a rotary evaporator to give comparison fullerene composite (7).

Hexane (200 parts) was added to this comparison fullerene composite (7), and the mixture was stirred. As a result, the comparison fullerene composite was scarcely dissolved, and a black insoluble material was precipitated.

### Comparative Example 14

A polymer dispersant (20.0 parts, manufactured by Ajinomoto-Fine-Techno Co., Inc., Ajisper PB821) was dissolved in toluene (200 parts). A solution obtained by dissolving C₆₀ (0.2 part, manufactured by Tokyo Kasei Kogyo Co., Ltd.) in toluene (200 parts) was added to this solution, and the mixture was stirred at room temperature for 20 min. The solvent was removed by a rotary evaporator to give comparison fullerene composite (8).

Hexane (200 parts) was added to this comparison fullerene composite (8), and the mixture was stirred. As a result, the comparison fullerene composite was scarcely dissolved, and a black insoluble material was precipitated.

### Examples 21-30

Compounds (1)-(10) obtained in Examples 1-10 were dissolved in hexane (500 parts) by 1.0 part each. A gas phase carbon fiber (0.5 part, manufactured by SHOWA DENKO K.K., VGCF-H) was added to each of these solutions, and the gas phase carbon fiber was dispersed by ultrasonication for 30 min. As a result, the gas phase carbon fiber was dispersed finely, and the dispersion remained black 1 hr later.

### Comparative Example 15

A gas phase carbon fiber (0.5 part, manufactured by SHOWA DENKO K.K., VGCF-H) was added to hexane (500 parts), and dispersion of the gas phase carbon fiber was tried by ultrasonication for 30 min. As a result, the gas phase carbon fiber failed to finely disperse but almost all precipitated in about several seconds, making the solution colorless and transparent.

### Comparative Examples 16-20

The comparison compounds (1)-(5) (each 1.0 part) obtained in Comparative Examples 1-5 were dissolved in toluene (500 parts). A gas phase carbon fiber (0.5 part, manufactured by SHOWA DENKO K.K., VGCF-H) was added to each of these solutions, and dispersion of gas phase carbon fiber by ultrasonication for 30 min was tried. As a result, the gas phase carbon fiber failed to finely disperse but almost all precipitated in about 10 min when comparison compounds (1)-(4) were added and in about 30 min when comparison compound (5) was added, making the solutions colorless and transparent.

### Comparative Example 21

A titanate coupling agent (1.0 part, manufactured by Ajinomoto-Fine-Techno Co., Inc., Plenact KR TTS) was dissolved in hexane (500 parts). A gas phase carbon fiber (0.5 part, manufactured by SHOWA DENKO K.K., VGCF-H) was added to this solution, and dispersion of the gas phase carbon fiber was tried by ultrasonication for 30 min. As a result, the gas phase carbon fiber failed to finely disperse, but almost all precipitated in about 10 min, making the solution colorless and transparent.

### Comparative Example 22

An aluminate-based coupling agent (1.0 part, manufactured by Ajinomoto-Fine-Techno Co., Inc., Plenact AL-M) was dissolved in hexane (500 parts). A gas phase carbon fiber (0.5 part, manufactured by SHOWA DENKO K.K., VGCF-H) was added to this solution, and dispersion of the gas phase carbon fiber was tried by ultrasonication for 30 min. As a result, the gas phase carbon fiber failed to finely disperse, but precipitated in about 10 min, making the solution transparent.

### Comparative Example 23

A polymer dispersant (1.0 part, manufactured by Ajinomoto-Fine-Techno Co., Inc., Ajisper PB821) was dissolved in hexane (500 parts). A gas phase carbon fiber (0.5 part, manufactured by SHOWA DENKO K.K., VGCF-H) was added to this solution, and dispersion of the gas phase carbon fiber was tried by ultrasonication for 30 min. As a result, the gas phase carbon fiber failed to finely disperse, but precipitated in about 10 min, making the solution transparent.

### Example 31

Compound (10) (1.0 part) obtained in Example 10 was dissolved in cyclohexanon (500 parts). Phthalocyanine blue (0.5 part, manufactured by Dainichiseika Color & Chemicals Mfg. Co., Ltd.) was added to this solution, and phthalocyanine blue was dispersed by ultrasonication for 30 min. As a result, phthalocyanine blue dispersed fine, and did not sediment even one week later.

### Comparative Example 24

A commercially available pigment dispersant (1.0 part, manufactured by Ajinomoto-Fine-Techno Co., Inc., Ajisper PB821) was dissolved in cyclohexanon (500 parts). Phthalocyanine blue (0.5 part, manufactured by Dainichiseika Color & Chemicals Mfg. Co., Ltd.) was added to this solution, and dispersion of phthalocyanine blue was tried by ultrasonication for 30 min. As a result, phthalocyanine blue dispersed immediately after ultrasonication but sedimented 24 hr later.

### Comparative Example 25

Phthalocyanine blue (0.5 part, manufactured by Dainichiseika Color & Chemicals Mfg. Co., Ltd.) was added to cyclohexanon (500 parts), and dispersion of phthalocyanine blue was tried by ultrasonication for 30 min. As a result, phthalocyanine blue sedimented when 30 min lapsed from ultrasonication.

### Example 32

The solvent was evaporated by a rotary evaporator from the dispersion of phthalocyanine blue in cyclohexanon obtained in Example 31 to give a surface-treated phthalocyanine blue (1) (1.5 parts). This surface-treated phthalocyanine blue (1) was added to xylene (500 parts), and phthalocyanine blue was dispersed by ultrasonication for 30 min. As a result, phthalocyanine blue dispersed fine, and did not sediment even one week later.

### Comparative Example 26

The solvent was evaporated by a rotary evaporator from the dispersion of phthalocyanine blue in cyclohexanon obtained in Comparative Example 25 to give a surface-treated phthalocyanine blue (2) (1.5 parts). This surface-treated phthalocyanine blue (2) was added to xylene (500 parts), and dispersion of phthalocyanine blue was tried by ultrasonication for 30 min. As a result, phthalocyanine blue dispersed fine immediately after ultrasonication, but sedimented 12 hr later.

### Comparative Example 27

Phthalocyanine blue (1 part) was added to xylene (500 parts), and dispersion of phthalocyanine blue by ultrasonication for 30 min was tried. As a result, phthalocyanine blue sedimented in 30 min from ultrasonication.

From the foregoing, a compound having a calixarene skeleton, a titanate coupling agent, an aluminate-based coupling agent, a polymer dispersant and a pigment dispersant, each being free of the characteristics of the present invention, cannot disperse or solubilize carbon fiber, fullerene and phthalocyanine blue in an organic solvent. It is clear, however, that the use of a dispersant or a solubilizer containing the calixarene compound (I) of the present invention enables dispersion or solubilization.

### INDUSTRIAL APPLICABILITY

The calixarene compound (I) of the present invention can improve affinity of objects such as a carbon-based material (e.g., fullerene, carbon fiber and the like), an organic pigment (e.g., phthalocyanine blue and the like), and the like for an organic solvent and affinity of resin, lubricant and the like for an organic matrix, whereby the objects can be dispersed or solubilized.

This application is based on a patent application No. 316869/2002 filed in Japan, the contents of which are hereby incorporated by reference.

1. A calixarene compound, wherein, of the phenolic hydroxyl groups constituting calixarene,
(A) at least one is not substituted, and
(B) at least one is substituted by a group having the total carbon number of not less than 10, which comprises a group comprising one or more alkyleneoxy groups and/or a hydrocarbon group.

2. The calixarene compound of claim 1, wherein the calixarene compound is represented by the following formula (1) or (2): 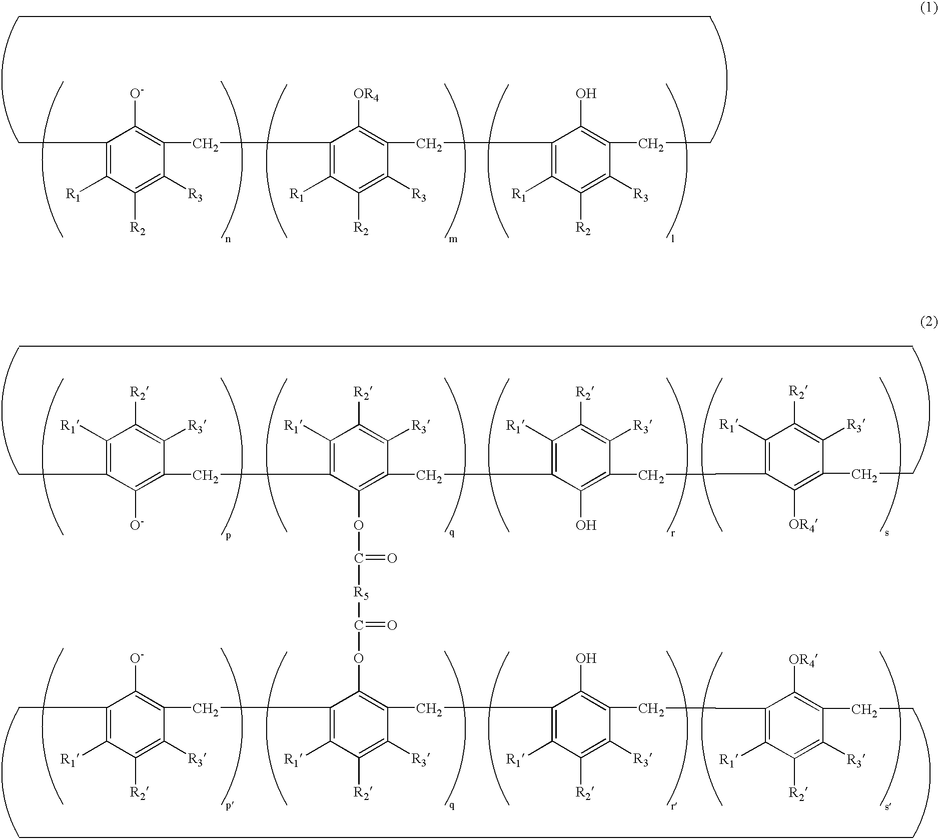
wherein R₁, R₂, R₃, R₁′, R₂′ and R₃′ may be the same or different and each is a hydrogen atom, a chain hydrocarbon group optionally having substituent(s), an aryl group optionally having substituent(s), an alkoxy group optionally having substituent(s), a halogen atom, a nitro group, an acyl group, a carboxyl group, a sulfonic acid group or an amino group optionally having substituent(s),
R₁, R₂ and R₃ in the number of n, m or 1 may be the same or different,
R₁, R₂′ and R₃′ in the number of p, q, r or s may be the same or different,
R₁′, R₂′ and R₃′ in the number of p′, r′ or s′ may be the same or different;
R₄ and R₄′ may be the same or different and each is a C₁₀₋₂₀ alkyl group optionally having substituent(s) or a C₉₋₂₀ alkyl-carbonyl group optionally having substituent(s) or a group represented by the formula (3): —(R₆CO₂)x-R₇, the formula (4): —(R₈O)y-R₉ or the formula (5): —(CO—R₁₀O)w-COR₁₁ (in the formula (3), the formula (4) and the formula (5), R₆, R₉ and R₁₀ may be the same or different and each is a C₁₋₂₀ alkylene group optionally having substituent(s), R₇, R₉ and R₁₁ may be the same or different and each is a hydrogen atom, an acyl group or a C₁₋₂₀ alkyl group optionally having substituent(s), and x, y and w may be the same or different and each is an integer of 1 to 200) (provided that the total carbon number of each of the groups represented by the formula (3), the formula (4) and the formula (5) is not less than 10),
R₄ in the number of m may be the same or different,
R₄′ in the number of s may be the same or different,
R₄′ in the number of s′ may be the same or different;
R₅ is a C₂₋₂₀ alkylene group optionally having substituent(s),
R₅ in the number of q may be the same or different;
n is an integer of 0 to 8, m is an integer of 1 to 9, 1 is an integer of 1 to 9, provided that n+m+1 is an integer of 4 to 10;
p and p′ may be the same or different and each is an integer of 0 to 7, q, r, r′, s and s′ may be the same or different and each is an integer of 1 to 8, provided that p+q+r+s and p′+q+r′+s′ may be the same or different and each is an integer of 4 to 10.

3. The calixarene compound of claim 2, wherein R₁, R₂, R₃, R₁′, R₂′ and R₃′ may be the same or different and each is a hydrogen atom or a chain hydrocarbon group optionally having substituent(s).

4. The calixarene compound of claim 2, wherein R₄ and R₄′ may be the same or different and each is a C₁₀₋₂₀ alkyl group optionally having substituent(s), a C₉₋₂₀ alkyl-carbonyl group optionally having substituent(s) or a group represented by the formula (3): —(R₆CO₂)x-R₇ or the formula (4): —(R₈O)y-R₉ (in the formula (3) and the formula (4), R₆ and R₈ may be the same or different and each is a C₁₋₂₀ alkylene group optionally having substituent(s), R₇ and R₉ may be the same or different and each is a hydrogen atom, an acyl group or a C₁₋₂₀ alkyl group optionally having substituent(s), and x and y may be the same or different and each is an integer of 1 to 200) (provided that the total carbon number of each of the groups represented by the formula (3) and the formula (4) is not less than 10).

5. The calixarene compound of claim 4, wherein R₁, R₂, R₃, R₁′, R₂′ and R₃′ may be the same or different and each is a hydrogen atom or a chain hydrocarbon group optionally having substituent(s).

6. A composite comprising the calixarene compound of claim 1 and a material that differs from the calixarene compound.

7. The composite of claim 6, wherein the different material is a carbon-based material.

8. The composite of claim 7, wherein the carbon-based material is carbon black, carbon nano tube, graphite, carbon fiber, amorphous carbon, or diamond powder.

9. The composite of claim 7, wherein the carbon-based material is fullerene.

10. The composite of claim 6, wherein the different material is an organic pigment.

11. The composite of claim 10, wherein the organic pigment is a phthalocyanine pigment, an azo pigment, a quinacridone pigment, a diketopyrrolopyrrole pigment, or an anthraquinone pigment.

12. The composite of claim 6, wherein the different material is a lubricant.

13. A composition comprising the calixarene compound of claim 1, a material that differs from the calixarene compound, and an organic solvent or matrix.

14. The composition of claim 13, wherein the different material is a carbon-based material.

15. The composition of claim 14, wherein the carbon-based material is carbon black, carbon nano tube, graphite, carbon fiber, amorphous carbon, or diamond powder.

16. The composition of claim 14, wherein the carbon-based material is fullerene.

17. The composition of claim 13, wherein the different material is an organic pigment.

18. The composition of claim 17, wherein the organic pigment is a phthalocyanine pigment, an azo pigment, a quinacridone pigment, a diketopyrrolopyrrole pigment, or an anthraquinone pigment.

19. The composition of claim 13, wherein the different material is a lubricant.

20. A method of dispersing or solubilizing a substance comprising (a) providing a calixarene compound of claim 1 and (b) combining the calixarene compound with an organic solvent or matrix and a substance that differs from the calixarene compound and the organic solvent or matrix and that is desired to be dispersed or solubilized in the organic solvent or matrix, such that the calixarene compound acts on the surface of the different substance, thereby dispersing or solubilizing the substance in the organic solvent or matrix.

21. The method of claim 20, wherein the different substance is a carbon-based material.

22. The method of claim 21, wherein the carbon-based material is carbon black, carbon nano tube, graphite, carbon fiber, amorphous carbon, or diamond powder.

23. The method of claim 21, wherein the carbon-based material is fullerene.

24. The method of claim 20, wherein the different substance is an organic pigment.

25. The method of claim 24, wherein the organic pigment is a phthalocyanine pigment, an azo pigment, a quinacridone pigment, a diketopyrrolopyrrole pigment, or an anthraquinone pigment.

26. The method of claim 20, wherein the different substance is a lubricant.
